(19) United States
(12) Patent Application Publication
(10) Pub. No.: US 2012/0244212 A1
(43) Pub. Date: Sep. 27, 2012

(54) ENHANCED METHOD AND COMPOSITION FOR THE TREATMENT OF HIV+ TUBERCULOSIS PATIENTS WITH ANTI-RETROVIRAL DRUGS AND LIPOSOMAL ENCAPSULATION FOR DELIVERY OF REDUCED GLUTATHIONE

(76) Inventor: Frederick Timothy Guilford, Palo Alto, CA (US)

(21) Appl. No.: 13/458,449

(22) Filed: Apr. 27, 2012

Related U.S. Application Data
(63) Continuation-in-part of application No. 12/065,753, filed on Sep. 3, 2008.
Application No. 12/065,753 is a continuation-in-part of application No. 11/163,979, filed on Nov. 6, 2005.
(60) Provisional application No. 60/522,785, filed on Nov. 7, 2004.

(51) Int. Cl. A61K 9/127; A61P 31/06; A61P 31/14; A61K 31/195

(52) U.S. Cl. 424/450; 514/562

(57) ABSTRACT

The invention is the use of a therapeutically effective amount of glutathione (reduced) in a liposome encapsulation for oral administration to improve symptoms of illnesses that are related to tuberculosis and HIV and more generally viruses and for the treatment and prevention of virus, particularly HHV-6 and EBV, which liposomal encapsulation of glutathione (reduced) is referred to as liposomal glutathione. The application references specifically reduced glutathione and its importance, and how to stabilize it effectively so it can be taken orally, and need not be refrigerated. New uses for tuberculosis are discussed. The combination is proposed of reduced glutathione and Highly Active Anti-Retroviral Therapy having at least one pharmaceutical composition selected from the group of Nucleoside/tide Reverse Transcriptase Inhibitors (NRTIs), Protease Inhibitors (PIs), and Non-nucleoside Reverse Transcriptase Inhibitors (NnRTIs), and further anti-tuberculosis drugs.

### CONTINUATION DATA

For U.S. purposes, this application claims benefit of, and as required, is a continuation-in-part of U.S. Provisional Application 60/596,171 filed on Sep. 6, 2005 with the same name as this application, and of U.S. Provisional Application 60/824,671 filed on Sep. 6, 2006 of this name, and is intended to be a continuation-in-part or the substantive equivalent in any regional or national stage in which continuation is permitted to preserve an earlier filing date, and is a continuation in part of U.S. application Ser. No. 12/065,753, and pending application Ser. No. 11/163,979 filed Nov. 6, 2005 claiming priority from 60/522,785 filed Nov. 7, 2004, which applications are adopted by reference

### SUMMARY OF INVENTION

The invention is the use of a therapeutically effective amount of glutathione (reduced) in a liposome encapsulation for oral administration and anti-tuberculosis compositions, and for HIV+ patients, anti retroviral pharmaceutical compositions to improve symptoms of illnesses that are related to these diseases, which liposomal encapsulation of glutathione (reduced) is referred to as liposomal glutathione.

### TECHNICAL FIELD

The invention relates to the field of delivery of a nutrient substance, glutathione, in the biochemically-reduced form (“reduced glutathione”) to tissue sites such as brain and components of the immune system such as the macrophage using liposomal encapsulation to both maintain glutathione in the reduced state and to increase the delivery of reduced glutathione to sites of infection by viruses and bacteria. The delivery is accomplished in a liposome encapsulation via absorption across the mucosa of the nose, mouth, gastrointestinal tract, or after topical application for transdermal, or by intravenous infusion.

### GENERAL BACKGROUND

The tripeptide L-glutathione (GSH) (gamma-glutamyl-cysteinyl-glycine) is well known in biological and medical studies to serve several essential functions in the cells of higher organisms such as mammals. It is functional when it appears in the biochemical form known as the reduced state (GSH). When oxidized, it forms into a form known as a dimer (GSSG). Glutathione is not considered an essential nutrient, which means that it is normally formed in adequate amounts in the body from the combination of its amino acid components, glycine, glutamine and cysteine. The biosynthesis of reduced glutathione (GSH) depends on the enzyme gamma-glutamylcysteine synthetase to combine cysteine and glutamine and GSH synthetase to add the glycine to the first two amino acids. The availability of cysteine has been shown to be the component that limits the production of glutathione (Bender, O'Connor).

While there is ample discussion of glutathione generally, the literature has not discussed glutathione in the reduced state (GSH) which functions as an important antioxidant, protecting cells against free-radical mediated damage, a detoxifying agent by transporting toxins out of cells and out of the liver, and as a cell signal by controlling the oxidative state, particularly in the immune system.

Glutathione has been shown to diminish the replication of virus such as influenza and HIV in cell culture. At the same time, delivery of glutathione to the human system has been problematic as the use of glutathione in its pure powdered form has been shown to be not effectively absorbed (Witschi, 1992). While the oral administration of plain glutathione for oral administration has been referenced by Jones et al in U.S. Pat. No. 6,107,281 there was no reference to the delivery of glutathione in a liposome, and no reference to reduced glutathione and its importance, and how to stabilize it effectively so it can be taken orally, and need not be refrigerated.

The use of the term “glutathione” or “glutathione (reduced)” in this application will refer to glutathione in the reduced state.

Human Herpes Virus 6 (HHV-6) is a member of the herpes family of viruses. The virus was discovered in 1986 in individuals with disorders with an overproduction of white blood cells from sites such as lymph nodes, spleen or thymus, sometimes referred to as lymphoproliferative disorders. (Salahuddin). HHV-6 shows a widespread distribution as it is a cause of a common childhood disease, roseola, that affects 95% of children. Roseola (also known as sixth disease, exanthem subitum, and roseola infantum), most commonly affects children between the ages of 6 months and 2 years. The course of the illness usually included several days of high fever, followed by a distinctive rash just as the fever breaks. The virus belongs to the herpes family of viruses, but is not associated with skin sores, but as with other members of the herpes virus family, it can develop a life-long persistence in a dormant state.

In spite of the fact that the HHV-6 virus is frequently reactivated during other illnesses, it appears to remain unapparent clinically unless there is some concurrent event that diminishes the immune defense of the individual. Even when activated, HHV-6 has been thought to be only a contributor to the pathogenicity of other viruses or existing autoimmune disease, rather than a direct pathogen itself (Krueger).

While its role as a common infection of childhood suggests that HHV-6 is only rarely a source of serious problem, HHV-6 is becoming recognized as a significant pathogen for organ transplant recipients and there is increasing evidence that it may play a role in central nervous system disease (De Bolle). While the role of HHV-6 as a primary pathogen is still controversial, there is also a growing amount of evidence suggesting HHV-6 is a pathogen and this significance is only just begun to be appreciated.

In the immune system the type of white blood cell called lymphocytes have been found to perform different functions in immune defense. Before the function of these cells was understood, a way to identify the cells was found using antibodies specific to various clusters of proteins found on the surface of the lymphocyte. These antibodies were able to chart the different types of lymphocyte populations based on the appearance of specific immunologically distinctive protein clusters as markers. These protein markers ultimately were associated with functionally distinct populations of lymphocytes such as B-cells, helper T-cells (TH), cytotoxic T-cells (TC), and natural killer (NK) cells. These different populations have become designated by the cluster of differentiation (CD) antigen number. The first group identified was CD group 1, designated CD1. The second was designated CD2 and so on. At the time this designation was being formed, the actual function of the lymphocytes was not known. It has been subsequently shown that the white blood cells, called T helper (TH) lymphocytes always show a cluster designation number 4 and are now known as CD4. This marker shows up on the TH lymphocytes as well as monocytes and macrophage cells, but not on other lymphocytes. Cells that carry the CD4 proteins are also sometimes designated as CD4⁺ or CD4⁻ cells. Cytotoxic (that is toxic to cells) T cells or killer cells were found to have the designation CD8. The CD marker proteins have been found to play a role in viral infection. These proteins can be sites for viruses to attach to and enter cells. Different viruses are associated with the different CD markers. The proteins of the cluster designation 4, CD4, are important not only for designating the cells, but also because these proteins serve as a site of entry into the cell for the Human Immunodeficiency Virus.

Another herpes family virus, Epstein-Barr virus (EBV) that is associated with the common disease known as mononucleosis, attaches to the CD21 marker of B lymphocytes and enters the cell through this protein cluster. It is helpful to review what happens with Epstein-Ban virus infection as there are similarities with the cell machinery when HHV-6 infection occurs. However, one major difference is that the Epstein-Barr Virus (EBV) affects primarily B cells, while HHV-6 affects T cells more readily, due to the ability to enter cells through different cluster of difference markers, the CD markers. With both viruses, after the infection occurs, the immune system responds by sending T cells to inactivate the cells associated with the virus. With respect to EBV, this response of sending T cells to inactivate the cells is so strong and there is created such an excess of mononuclear T cells that EBV infections were originally known as mononucleosis. The name mononucleosis came from the fact that the mononuclear T cells were the most common cells seen on microscopic evaluation of the peripheral blood smears of individuals with EBV.

As the EBV virus enters the cell certain new proteins are made that can be recognized by the immune system. These proteins are called antigens. During the infection with EBV certain antigens are formed as the infection progresses. As EBV can cause both an acute disease and also form a low grade chronic infection, it is difficult to determine if the virus has become active or is in the chronic infection state. As the antigen level rises, the body creates antibodies against the antigens. The presence of antibodies against the increased level of antigens associated with the emerging viral infection have become valuable tools for determining whether an infection with EBV is new, past or has become reactivated. The antigens most frequently associated with a developing infection, whether it is new or recurrent, is the early antigen (EA). Both IgM, the acute phase antibody, or IgG, the antibody associated with chronic disease, can be formed against these resulting antigens which are created as a result of the viral infection.

The first antigen to appear during infection with EBV is the nuclear antigen (EBNA), however antibodies to this antigen do not appear until late in the infection. The early antigen of EBV as its name implies appears early in the infection and is produced during active viral replication. Thus, antibody to the early antigen (EA) can be used to detect active infection. Later in the infection, antibody to the capsule of the virus develops, called viral capsid antigen or EBV-VCA. With each of the antigens, the IgM develops early, but does not persist and the IgG develops later and is persistently elevated. The various combinations of the type of antibody, that is whether there is IgM or IgG and the type of antigen that they are specific for can then be used to determine if an infection with EBV is the initial infection, has occurred previously or is a recurrence of infection.

Because of the AIDS crisis resulting from Human Immunodeficiency virus (HIV), public awareness of the CD4 and CD8 cell markers on white blood cells has increased because CD4 and CD8 markers are often referred to in non-professional literature such as newspapers and magazines. CD4 and CD8 markers have become well-known as associated with the type of cells monitored during HIV infections. The T cell count sometimes referenced is referring to white bloods cells, and the white blood cells with the CD4 and CD8 markers are important indicators of the progress of the HIV virus. HIV virus uses the CD4 marker to enter the cell, thus this type of T cell becomes infected by HIV most readily. As the infection with HIV progresses the number of CD4 cells decreases more rapidly than other immune cells, as these are the cells that the virus enters most easily. The ratio between CD4 and CD8 cells has been used to monitor the progression of HIV disease. Because CD4 cells are involved in the coordination and stimulation of immune function, loss of CD4 cells results in decreased immune defense. In a normal situation the CD8 cells would be programmed by cytokines to attack and eliminate the viral infected CD4 cells. As the CD 8 cells are not infected directly by the HIV virus the CD8 cells creates a stable measurement of immune cells to compare the activity of the immune system against. The decrease in the number of CD4 cells relative to the number of CD8 cells is an indicator of progression of HIV disease.

Similarly, HHV-6 has an initial preference for the CD46 site on cells, especially in T cells. During childhood infection such as roseola, it has been shown that HHV-6 is most commonly recovered from the CD4+ cell population.

Four weeks following primary roseola infections virus could be recovered only from macrophages (Braun). The macrophage form of white blood cell plays a key role in immune function by engulfing foreign particles and organisms which are then carried to regional lymph nodes where the information is used to stimulate either T cell or B cell responses. Moreover, the macrophage engulfed, and inactivated foreign particles and organisms can then be excreted through the lymphatic system. However, as to roseola, macrophages have been considered a potential repository for latent infection. Because central nervous system cells called neurons and astrocytes located in the brain also carry the cluster designation number 46 or CD46 marker, they are also targets for HHV-6 and brain tissue has been shown to be an potential target for both the active and latent infections with HHV-6 (De Bolle).

Current management of HHV-6 infection relies on antiviral medications, but those medications have not demonstrated significant success. In vitro studies have shown that HHV-6 is relatively resistant to acyclovir, a medication commonly used to treat herpes type infections. The resistance to acyclovir is consistent with the fact that the HHV-6 virus does not encode a thymidine kinase. In vitro studies do suggest that the virus is sensitive to ganciclovir and phosphonoformic acid (foscarnet) and cidofovir (Dockrell). However gangciclovir has limitations which include a dose related decrease in the white blood cell count, which may be irreversible, and a potential for loss of platelets (De Bolle) Foscarnet is also limited as it has a dose dependent kidney toxicity; Cidovir has a similar kidney toxicity (De Bolle. Thus, therapeutic choices for the management of HHV-6 are limited.

The present invention proposes a novel approach for the management of tuberculosis, including for HIV+ patients, using a liposome to deliver reduced glutathione to sites of viral infection in combination with anti-tuberculosis drugs, and for HIV+ patients, in combination with anti-retroviral drugs. The application proposes a novel mechanism of action of the disclosed combination that stabilizes infected cells during viral infection resulting in surprisingly higher cell survival in in-vitro demonstrations of the anti-viral effect of the present invention

Oxidation stress occurs when the balance between the production and the disposal of reactive oxygen species (ROS) favors the production of excess ROS, also known as free radicals. Many viral infections involve a change in the machinery of the cell designed to produce more virus, but at the same time creates oxidation stress, an injury to the cell that results in a marked depletion of extra- and intracellular GSH levels.

A liposome is a microscopic fluid-filled pouch whose walls are made of one or more layers of phospholipid materials identical to the phospholipid that makes up cell membranes. Liposomes could be referred to as nanoscopic, i.e. on the order of one-billionth in size. The liposomes used in the present invention are between 100 and 500 nanometer in size. That small size enables liposomes to pass through many cell walls and chemical pores (like a chemical hole), which penetration of a cell could not occur if the substance was not contained in a liposome. In addition, liposomes are known to fuse with cells and to deliver their contents into the cell (Constantinescu). Lipids can be used to deliver materials such as drugs to the body because of the enhanced absorption of the liposome. The outer wall of the liposome is fat soluble, while the inside is water-soluble. This combination allows the liposome to become an excellent method for delivery of water-soluble materials that would otherwise not be absorbed into the body. A common material used in the formation of liposomes is phosphatidylcholine, the material found in lecithin. A more detailed description of the constituents of this invention is provided.

Replacing glutathione in human deficient states has been difficult because of the lack of direct absorption of glutathione after oral administration. Glutathione is a water-soluble peptide. Glutathione is very temperature sensitive, and easily scavenged, or chemically converted from its glutathione reduced state. This characteristic of glutathione is thought to prevent its absorption into the system after oral ingestion of glutathione. The fate of direct oral ingestion of glutathione has been demonstrated in a clinical study showing that 3 grams of glutathione delivered by oral ingestion does not elevate plasma glutathione levels (Witschi, 1992).

This invention proposes the use of the liposome encapsulation of reduced glutathione to enable restoration of glutathione to the body, particularly in those tissues that have become deficient. The invention also overcomes the well-known blood-brain barrier that has inhibited the uptake into brain tissue of traditional medicaments and traditional means of administration.

Liposomes are particularly useful in HHV-6 type infection as they have been shown to have both a preferential uptake by macrophages (Van Rooijen), but also show preferential concentration in the brain of experimental animal models with brain inflammation, such as a model mimicking multiple sclerosis (Schmidt).

While the concept that the use of glutathione in the reduced state, as a general matter, will inhibit viral replication has been referenced previously, there is no reference to the use of oral or intravenous liposome encapsulated glutathione in the reduced state for the treatment of viral infections, and particularly HIV+ viral infections where there is tuberculosis present.

In the practice of the present invention the combination of using liposomes encapsulating reduced glutathione presents several advantages that have previously not been reported in a product. These advantages include:
1. Liposomes that are stable for an extended period of up to two years in a liquid state at room temperature without refrigeration.
2. Liposomes that are capable of stabilizing glutathione in the reduced state in the product container at room temperature for an extended period of time.
3. Liposomes that are capable of maintaining glutathione in a reduced state after oral ingestion. The fate of orally ingested liposomes and their ability to function has been controversial according to Smith in U.S. Pat. No. 6,764,693. While Smith references the use of oral ingestion of liposomes, there is no reference to the use of an oral liposome containing only reduced glutathione. The present application also contains demonstrations of clinical efficacy of the liposomal encapsulation of reduced glutathione in the examples cited.
4. The encapsulation of reduced glutathione in a liposome allows the preferred delivery of the product to macrophages that are frequently involved as the site of active and latent infection with viruses, such as HHV-6.
5. Liposomes are capable of passing through the blood brain barrier to carry glutathione to affected brain cells.
6. Liposomes are known to be taken up or preferentially at sites of inflammation (Awashti, 1998, 2002).
   Liposomal encapsulation of reduced glutathione has been determined to be stable for at least 14 months without refrigeration and remains capable of anti-viral effect. See Example in Preferred Embodiments at “LIPOSOMAL GLUTATHIONE ANTIVIRAL EFFECT ON HHV-6 INFECTED CELL CULTURE.”

The combination of these attributes in the present invention creates a therapeutic advantage that is novel, and accomplishes an advantageous result in a new way. In the practice of the present invention the active agent, reduced glutathione, is rendered available systemically from dermal (in some situations), oral or nasal administration, and is carried to the sites of inflammation, which occur with activation of viruses such as HHV-6 and results in an antiviral effect.

In addition to the antiviral effect, the invention modulates the “cytokine storm” described by Osterholm in his article describing the deleterious effects of influenza. While the concept of damage from virus has been the damage to infected cells, it is now becoming accepted that more damage occurs from the release of cytokines in response to virus. As reviewed in the provisional patent by Guilford Ser. No. 60/594,324 on 2005-03-29 entitled “Administration Of Glutathione (Reduced) Via Intravenous Or Encapsulated In Liposome For The Amelioration Of Flu-Like Viral Symptoms And Treatment And Prevention Of Virus” the most severe damage from viruses like influenza come from the release of cytokines. Although the natural defense design appears to be intended to stop virus attachment and infection, the release of cytokines requires that the system from the cell level to the systemic level have the ability to modulate and moderate the effect that cytokines like tumor necrosis factor (TNF) have on the cells of the entire system. As the cytokines are released, if their effect is not modulated, a cascading of negative effects can occur, which is termed the cytokine storm. The effects of the cytokine storm lead to the sudden morbidity and mortality of viral infection. When the system is working properly, viruses such as influenza should be self limiting. As cytokines are released, an increase in free radical production occurs with the potential to develop what is called a free radical cascade. This leads to the oxidative stress that accompanies viral infection and allows the progression of viral infection. A system deficient in glutathione, at either the cellular or systemic level, is more susceptible to damage from the cytokine storm as well as the cascade of free radicals and oxidation stress. It is the intention of the present invention to ameliorate the damage from both cytokine release and oxidation stress at the cell level, stabilize membranes at both the cell and systemic level, and modulate the release of cytokines to moderate the damage from these mechanisms that result in the morbidity and mortality from viral infection such as HHV-6.

### Background of Immune Function

A synergistic effect related to co-infection of HHV-6 with other organisms, including bacterial infections such as Legionella (Russler) and mycoplasma (Nicolson), has been reported. HHV-6 infection has also been associated as a potential cofactor in the pathogenesis of a number of serious diseases, including HIV (Ablashi, 1995). The effect of HHV-6 in potentiating additional infections is thought to be mediated by an immunosuppressive effect of the HHV-6 virus.

To understand the interaction of HHV-6 and immune function some basic immunology is needed.

In general, the immune systems has involves two mechanisms, non-specific immunity and specific immunity. The two systems interact and influence each other.

Nonspecific or innate immunity is considered to be an older system in terms of evolutionary development, is present at birth and does not require a previous encounter with an offending substance to stimulate an action. In the context of innate immunity barriers such as skin and secretion of gastric acid are mentioned as protectants. Included in innate immunity are two cell components, (1) the phagocyteic system, which ingests and digests invading organisms and (2) the natural killer (NK) cells. NK cells function to kill certain cells such as tumors, microorganisms and cells infected with virus. There are also soluble components of innate immunity, which include proteins, and cytokines. The cells in the immune system associated with surveillance that is the recognition and destruction of abnormal cells such as cancer cells and cells containing viruses. The cells that perform this function as an innate function of the cell are called Natural Killer or NK Cells. NK Cells originate in the bone marrow and are distributed throughout the body. The largest number are found in the peripheral blood system, followed by the number found in the spleen, and the number found in lymph nodes (Uchida).

Cells that ingest foreign particles and invading organisms included neutrophils and monocytes, white blood cells with a single nucleus which describes lymphocytes and macrophages in the blood and the macrophages, which are found in primarily in tissues Macrophages are generally found at the interface of tissues with blood such as the vascular system or cavitary spaces such as the lung, liver (Kupfer cells), joint cavities, and the perivascular microglial cells lining the central nervous system, and kidneys. Again, the macrophage plays a key role in immune function by engulfing foreign particles and organisms which are then carried to regional lymph nodes where the information is used to stimulate either T cell or B cell responses.

Adaptive or Specific immunity is characterized by learning, adaptability and memory. The cellular components are the lymphocytes and the soluble components are immunoglobulin such as Immunoglobulin G (IgG) The peripheral blood monocytes called lymphocytes are divided into two subsets, those formed or influenced by a passage through the thymus gland called T cells and those originating in the bone-marrow, or B cells.

B cells can be formed that are specific in their ability to recognize any number of antigens and are able to recognize the various antigens by their surface receptors called surface immunoglobulins. After an antigen binds to a surface immunoglobulin, a series of events including proliferation and differentiation of that B cell results in secretion of Immunoglobulin that is the specific antibody for that antigen. This type of reaction which forms immunoglobulin to a particular antigen is what happens with allergy, such as specific antibody to an allergy antigen or after immunization with a vaccine. The presence of the antibody specific to an antigen is a way of recognizing an immune response to the material. It is also typical of a form of immune response typified by the production of cytokines that create this response and is typically referred to as the T Helper Cell 2 response (TH2).

T cells recognize Antigens by a surface receptor called the T-cell receptor or TCR. Lymphocytes are characterized by having a protein unit called the TCR associated with a molecule called CD3. The whole unit is called the TCR/CD3 complex and the CD3 molecule is stable and a marker for the general group of circulating cells called lymphocytes.

Receptors are displayed on the surface of lymphocytes. receptors that are more variable and help categorize subtypes of these cells. The T Cell receptors are variable, and these have characteristics depending on what are called clusters of differentiation or CD that is typical of various cell types Because the T cell receptors are formed by various genes, which were given the names alpha, beta, gamma and delta genes when they were first identified. These genes are often represented by the lower case Greek letters for each: α, β, γ, and δ. T cells are first divided according to the combination of these genes that they express and thus form two groups, the αβ and γδ T cell lineages. The αβ T-cells subsequently divide further into the T cells known as CD4+ and CD8+ T cells. During normal immune development, maturation of these cells includes a process that selects out the CD4 and CD8 cells that would react to normal tissues occurs, leaving T cells that respond only to foreign proteins or antigens.

The cluster of differentiation called CD4 ultimately turned out to form the T-Helper cells. The CD cells labeled CD8 turned out to have characteristics now known as T-Suppressor cells. The cells that did not differentiate into a labeled variety but carried the γδ gene and are called γδT cells are is still being investigated. They are thought to provide immune response to specific invading organisms such as viruses like HHV-6 and other invaders including bacteria. (Lusso). When the γδT cell line is lost, such as can happen during infection with HHV-6, there is an increased likelihood of severe or persisting infection with HHV-6 and also other impairments of immune defense.

Cytokines are small protein-like molecules called polypeptides that are secreted from monocytes and lymphocytes after interaction with a variety of materials such as antigens, toxins or even other cytokines. As they circulate locally as well as systemically through the blood they function like immune hormones. Cytokines affect the magnitude of inflammation or immune responses. While they can be released by lymphocyte interaction with a specific antigen, they can be released by non-specific antigens. Thus cytokines bridge both the innate and adaptive immune systems.

The type of response to immune challenge is determined by the cytokines that are released during the challenge. The T cells called helper cells determine this response based on the cytokines that they release. For the purpose of description of activity the response stimulated by the TH cells is referred to as being of two types, TH1 and TH2. The TH1 pattern is characterized by the release of interleukin-12 (IL-12) and interferon γ (IFN-γ) production. These cytokines increase the cell-mediated immunity. The TH2 response characterized by IL-4 and IL-10 production and the upregulation of the production of antibodies such as Immunoglobulins G and E (IgG and IgE). The cytokines related to the two different responses tend to each down regulate the other. For example IFN-γ inhibits TH2 associated cytokine production and IL's 4 and 10 inhibit TH1 associated function. When the balance between TH1 and TH2 responses reaches an extreme the ability to overcome infection either locally or through the whole body is impaired (Peterson).

The cells which are responsible for presenting antigenic material to the lymph nodes and in determining whether the immune system responds with TH1 responses or TH2 responses are called Antigen Presenting Cells (APC). These cells include macrophages, B lymphocytes and dendritic cells. These types of cells are present in tissues which come in contact with the environment such as skin, nose, lungs, stomach and intestines. The name dendritic cell initiates from their appearance as they have elongated, somewhat spiky looking arms called dendrites. They look somewhat like a type of nerve cell that connect to the next nerve down the line. These extensions are called dendrites. The function of the dendrites on these immune cells is to allow a single cell to come in contact with a large number of other cells at one time. Dendritic cells and the other antigen presenting cells carrying antigenic material can migrate to lymph nodes and activate helper T-cells, killer-T cells as well as B-cells. A lack of glutathione in the antigen presenting cell (APC) will result in an inhibition of the TH1 cytokine production in favor of a TH2 response (Peterson). This response has been shown to be reversible in vitro. An object of the current invention is to reverse the TH2 predominance in-vivo, that is in the mammalian system, with the resulting resolution of chronic inflammation and restoration of the balance between the two systems. As the APC's engulf particles of the size of the liposomes used in the present invention, reduced glutathione can be delivered to these cells and create a more efficient immune function with resolution of symptoms related to diseases characterized by chronic inflammation.

The response of Th2 is to cause production of more immunoglobulins and to release cytokines which cumulatively create constriction of the local blood vessels to promote release of extra-cellular fluids and to summon additional lymphocytic cells. The combination of these actions serves to dilute out or wall off both the injurious agent and the injured tissue. While this is useful to contain the initial exposure to an invader, the persistence of this response will lead to tissue damage. When the reaction persists and damage to tissues occurs, the reaction is called chronic inflammation. The redness, soreness and heat, responses which in medical terms are known as rubor, dolor and color respectively, are typical of inflammation particularly of the TH2 response. In a balanced immune response, with adequate glutathione available, the TH1 cell mediated cytokines are also released, and are able to clean up, kill and remove the invading microbe reducing the time of inflammatory interaction and lessening the chance of chronic inflammation developing. The coordinated interaction of the both of the TH1 and TH2 systems also leads to the efficient removal of viral invaders. When the efficiency of the Th1 system is decreased and the TH2 system is correspondingly increased the effect is a continued release of inflammatory mediators. When this response causes tissue damage, it is referred to as chronic inflammation. Thus, the term “chronic”, while generally connoting the passage of time can also occur in the short period of time associated with the onset of a virus, if the balance between the two categories of immune response is uncontrolled. The ability to aid the correction of this loss of balance and coordination that occurs during inflammatory reactions and results in tissue damage is the focus of the present invention. The use of the liposomal encapsulated reduced glutathione allows the rapid return of control to a system that has been “cascading out of control”.

A cytokine that has been shown to be increased after infection with HHV-6 is Tumor necrosis factor α (TNF-α). TNF-α shares many biological activities with another cytokine called IL-1β. Both of these cytokines cause the type of heat and pain associated with inflammation. In addition TNF-α is associated with the activation of T lymphocytes, as well as stimulation of fibroblast proliferation and neutrophil activation. TNF also has the ability to encourage the formation of toxic forms of oxygen, called reactive oxygen species (ROS) that are capable of destroying microorganisms such as viruses. TNF-α is produced by activated macrophages, T and B lymphocytes, natural killer cells, astrocytes, endothelial cells, smooth muscle cells, some tumor cells, and epithelial cells. TNF-α is produced in response to infections as part of the normal response to infection from both virus and bacteria (Gomez), as well after noxious insult such as toxin exposure. Glutathione is required for defense in each of these situations, and with the presence of these responses a greater pressure is placed on the availability of glutathione to stabilize tissues exposed to TNF-α

TNF-α has found to be elevated in individuals with HIV and thought to be due to the activation or stimulation of the production of products from the cells such as lymphocytes producing massive amounts of TNF-α. This has been observed particularly in individuals with HIV, resulting in the classic wasting syndrome that accompanies HIV infection (Shikuma). An object of the present invention is to provide reduced glutathione to the sites of inflammation that are producing excessive amounts of TNF-α and to counter these effects resulting in a return to more normal weight, the present invention helps to stabilize cells against the deleterious effects of TNF-α and the resulting wasting syndrome associated with chronic infection such as HIV.

In addition, the present invention may be used in conjunction with the Highly Active Anti-Retroviral Therapies referred to as HAART or more simply anti-retroviral (ARV) pharmaceutical substances. These will collectively be called ARV drugs. Combination anti-HIV therapy is now the standard of care for people with HIV. Anti-HIV drugs fall into a number of main categories: Multi-class combination drugs, Nucleoside/tide Reverse Transcriptase Inhibitors (NRTIs), Protease Inhibitors (PIs), Non-nucleoside Reverse Transcriptase Inhibitors (NnRTIs), Immune-base therapies, Pharmacokinetic Enhancers, Fusion Inhibitors, Entry Inhibitors—CCR5 co-receptor antagonists, HIV integrase strand transfer inhibitors, Integrase Inhibitors, and Maturation Inhibitors.
Nucleoside/tide Reverse Transcriptase Inhibitors (NRTIs), which include abacavir (Ziagen), lamivudine, 3TC (Epivir), tenofovir (Viread), abacavir/lamivudine/zidovudine (Trizivir), lamivudine/zidovudine (Combivir), stavudine, d4T (Zerit), didanosine, ddI (Videx, Videx EC), zalcitabine, ddC (HIVID), and zidovudine, AZT (Retrovir).
Protease Inhibitors (PIs), which include amprenavir (Agenerase), nelfinavir (Viracept), saquinavir (Fortavase), indinavir (Crixivan), ritonavir (Norvir), saquinavir (Invirase), and lopinavir/ritonavir (Kaletra).
Non-nucleoside Reverse Transcriptase Inhibitors (NnRTIs), which include delavirdine (Rescriptor), efavirenz (Sustiva), and nevirapine (Viramune).

### Multi-Class Combination Drugs

| | Brand Name | |
|---|---|---|
| | (Trademark of | |
| | manufacturer | Manufacturer |
| Generic Name | in next column) | Name |
| efavirenz + tenofovir + emtricitabine | Atripla | Bristol-Myers |
| 600 mg of Sustiva (efavirenz), 300 | | Squibb and |
| mg of Viread (tenofovir DF) and 200 | | Gilead Sciences |
| mg of Emtriva (emtricitabine): one | | |
| pill once a day | | |
| Eviplera, rilpivirine + tenofovir + | Complera | Gilead Sciences |
| emtricitabine | | and Janssen |
| contains two different types of HIV | | Therapeutics. |
| drugs: one non-nucleoside reverse | | |
| transcriptase inhibitor (NNRTI) and | | |
| two nucleoside reverse transcriptase | | |
| inhibitors (NRTIs). | | |

### Nucleoside Reverse Transcriptase Inhibitors (NRTIs)

| | Brand Name | |
|---|---|---|
| | (Trademark of | |
| | manufacturer | Manufacturer |
| Generic Name | in next column) | Name |
| lamivudine and zidovudine | Combivir | GlaxoSmithKline |
| two drugs: 300 mg of Retrovir | | |
| (zidovudine) and 150 mg of Epivir | | |
| (lamivudine). BID | | |
| emtricitabine, FTC | Emtriva | Gilead Sciences |
| lamivudine, 3TC | Epivir | GlaxoSmithKline |
| abacavir and lamivudine | Epzicom | GlaxoSmithKline |
| zalcitabine, dideoxycytidine, ddC | Hivid | Hoffmann-La |
| (no longer marketed) | | Roche |
| zidovudine, azidothymidine, AZT, | Retrovir | GlaxoSmithKline |
| ZDV | | |
| abacavir, zidovudine, and | Trizivir | GlaxoSmithKline |
| lamivudine | | |
| tenofovir disoproxil fumarate and | Truvada | Gilead Sciences, |
| emtricitabine | | Inc. |
| enteric coated didanosine, ddI EC | Videx EC | Bristol Myers- |
| | | Squibb |
| didanosine, dideoxyinosine, ddI | Videx | Bristol Myers- |
| | | Squibb |
| tenofovir disoproxil fumarate, TDF | Viread | Gilead |
| stavudine, d4T | Zerit | Bristol Myers- |
| | | Squibb |
| abacavir sulfate, ABC | Ziagen | GlaxoSmithKline |
| RCV | Racivir | Pharmasset. It |
| | | prevents HIV |
| | | from entering the |
| | | nucleus of |
| | | healthy T-cells. |
| AMDX, DAPD | Amdoxovir | RFS Pharma |
| SPD754, AVX754) | Apricitabine | BioChem |
| | | Pharma −> Shire −> |
| | | Avexa. |
| ACH-126,443, Beta-L-Fd4C | Elvucitabine | Achillion |
| | | Pharmaceuticals |
| | GS 7340 | Gilead Science |

### Nonnucleoside Reverse Transcriptase Inhibitors (NNRTIs)

| | Brand Name | |
|---|---|---|
| | (Trademark of | |
| | manufacturer | Manufacturer |
| Generic Name | in next column) | Name |
| efavirenz + tenofovir* + | Atripla | Bristol-Myers |
| emtricitabine | | Squibb and |
| | | Gilead Sciences |
| rilpivirine + tenofovir* + | Complera | FTC Gilead |
| emtricitabine | | Sciences and |
| | | Janssen |
| | | Therapeutics |
| Rilpivirine | Edurant | Tibotec |
| | | Therapeutics |
| etravirine | Intelence | Tibotec |
| | | Therapeutics |
| delavirdine, DLV | Rescriptor | Pfizer |
| efavirenz, EFV | Sustiva | Bristol Myers- |
| | | Squibb |
| nevirapine, NVP | Viramune | Boehringer |
| | (Immediate | Ingelheim |
| | Release) | |
| nevirapine, NVP | Viramune XR | Boehringer |
| | (Extended | Ingelheim |
| | Release) | |
| | GSK-2248761 | ViiV |
| | | Healthcare. |
| UK-453061 | Lersivirine | ViiV |
| | | Healthcare. |

### Protease Inhibitors (PIs)

| | Brand Name | |
|---|---|---|
| | (Trademark of | |
| | manufacturer | Manufacturer |
| Generic Name | in next column) | Name |
| amprenavir, APV | Agenerase | GlaxoSmithKline |
| tipranavir, TPV | Aptivus | Boehringer |
| | | Ingelheim |
| indinavir, IDV, | Crixivan | Merck |
| saquinavir (no longer marketed) | Fortovase | Hoffmann-La |
| | | Roche |
| saquinavir mesylate, SQV | Invirase | Hoffmann-La |
| | | Roche |
| lopinavir and ritonavir, LPV/RTV | Kaletra | Abbott |
| | | Laboratories |
| Fosamprenavir Calcium, FOS-APV | Lexiva | GlaxoSmithKline |
| ritonavir, RTV | Norvir | Abbott |
| darunavir | Prezista | Tibotec, Inc. |
| atazanavir sulfate, ATV | Reyataz | Bristol-Myers |
| | | Squibb |
| nelfinavir mesylate, NFV | Viracept | Agouron |
| | | Pharmaceuticals |

### Immune-Based Therapies

These include Aralen (Chloroquine phosphate) DermaVir (therapeutic vaccine)—Genetic Immunity; Interleukin-7 (IL-7)—Cytheris; Lexgenleucel-T (VRX-496; gene therapy)—VIRxSYS; Plaquenil (hydroxychloroquine) Proleukin (aldesleukin, Interleukin-2, or IL-2) SB-728-T (gene therapy)—Sangamo Biosciences; Vacc-4x (therapeutic vaccine)—Bionor Pharma

### Pharmacokinetic Enhancers

| | Brand Name | |
|---|---|---|
| | (Trademark of | |
| | manufacturer | Manufacturer |
| Generic Name | in next column) | Name |
| ritonavir, RTV | Norvir | Abbott |
| | | Laboratories |
| GS-9350 | Cobicistat | Gilead Sciences |
| | SPI-452 | Sequoia |
| | | Pharmaceuticals |

### Fusion Inhibitors

| | Brand Name | |
|---|---|---|
| | (Trademark of | |
| | manufacturer | Manufacturer |
| Generic Name | in next column) | Name |
| enfuvirtide, T-20 | Fuzeon | Hoffmann-La |
| | | Roche & |
| | | Trimeris |

Entry Inhibitors—CCR5 co-receptor antagonist

| | Brand Name | |
|---|---|---|
| | (Trademark of | |
| | manufacturer | Manufacturer |
| Generic Name | in next column) | Name |
| maraviroc | Selzentry | Pfizer |
| | | binds to an entry |
| | | protein on the |
| | | membrane of |
| | | CD4 cells (CD4 |
| | | cells) called |
| | | CCR5 |
| enfuvirtide, ENF, T-20 | Fuzeon | |
| TBR-652, TAK-652 | Cenicriviroc | Tobira |
| | | Therapeutics |
| | | CD4 Entry |
| | | inhibitor |
| TNX-355 | Ibalizumab | Taimed Biologics |
| | PRO 140 | |

### HIV Integrase Strand Transfer Inhibitors

| | Brand Name | |
|---|---|---|
| | (Trademark of | |
| | manufacturer | Manufacturer |
| Generic Name | in next column) | Name |
| raltegravir | Isentress | Merck & Co., Inc. |
| GSK-572 | Dolutegravir | ViiV Healthcare |
| | | in collaboration |
| | | with Japan-based |
| | | Shionogi & Co. |
| | Elvitegravir | Gilead Science |

### Integrase Inhibitors

| | Brand Name | |
|---|---|---|
| | (Trademark of | |
| | manufacturer | Manufacturer |
| Generic Name | in next column) | Name |
| raltegravir | Isentress | Merck |
| GSK-572 | Dolutegravir | ViiV |
| | | Healthcare |
| GS-9137 | Elvitegravir | Gilead Science |
| Isentress | Raltegravir | Merck |
| | (RAL) | |

### Maturation Inhibitors

| | Brand Name | | |
|---|---|---|---|
| | (Trademark of | | |
| Generic | manufacturer | Manufacturer | |
| Name | in next column) | Name | Note |
| PA-457 | Bevirimat | Myriad Genetics | derivative of a |
| | | | Chinese herb |
| | | | called |
| | | | Syzigium |
| | | | claviflorum. |

This information is compiled from the Food and Drug Administration publications available on the Internet relating to HIV and AIDS Activities, from an organization called AIDSmeds based at 462 Seventh Avenue, 19th Floor, New York, N.Y. 10018-7424, also available on the Internet, and from AIDS information available at the U.S. National Institutes of Health setting out approved medicines.

It is an object of the present invention that liposomal glutathione be used as an adjunct to therapy with HAART (ARV) drugs. The advantage achieved with this novel combination is the improvement of immune function, stabilization of infected cells and amelioration of the oxidative effects of the HAART drugs while the therapy is proceeding. It is also an object of the invention to ameliorate the rate of progression of the oxidative stress induced vascular disease (Mondal) as well as other side-effects that are known to accompany HAART therapies (Montessori). The reader is referred to the Montessori article for a review of the dosing used for therapy of HIV and the side effects that may be seen in HIV therapy. While Mondal references the use of glutathione in in-vitro (laboratory cell culture studies) the lessen the effects of oxidation stress, the present invention of liposomal encapsulation of reduced glutathione represents a novel combination for the delivery of glutathione to the immune cells involved in both creating the oxidation stress and the tissues involved, the perivascular macrophages and peripheral blood mononuclear cells (PBMC). The type of vascular disease brought on by the oxidative stress induced by HAART therapy is an acceleration of the mechanisms of vascular disease in the general population not on HIV drug therapy. It is now commonly accepted that an inflammatory mechanism is associated with vascular disease. This inflammation is mediated by the same cells and inflammatory mechanism associated with the viral and intracellular bacterial infections discussed in this application. It is an object of the present invention for its use on a prolonged basis for the prevention and treatment of vascular disease. The unique attributes described for the present invention create a novel combination for the treatment of vascular disease. The dosing schedule for the treatment of vascular disease due is the same as that reviewed in the example of therapy for peripheral neuropathy in the example case 2.

There is also increasing evidence that defense against bacteria require the availability of reduced glutathione. For example, The activated macrophage the major phagocytic cell involved in the protection against infection with the organism Mycobacterium Tuberculosis, the cause of the Tuberculosis or Tb. Macrophages acquire the ability to kill the engulfed, and thus intracellular, pathogen after exposure to cytokines release by sensitized T lymphocytes. This event triggers mechanisms that are cidal, that is capable of killing, against bacteria. These bactericidal effects include the production of reactive oxygen species (ROS) known as free radicals, as well as the free radicals from nitric oxide species (RNS) formed. The generation of these toxic products is essential for the efficient function of cell-mediated immunity to intracellular infection. After invasion by or engulfing of a microbe the phagocyte releases ROS and RNS. Simultaneously, there is increased synthesis of GSH in order to protect the host cell from the toxic effects of ROS and RNS. Nitric oxide has been shown to react with glutathione, which creates S-nitrosoglutathione (GSNO). In turn GSNO can become a NO donor, which has been shown to inhibit growth of M. tuberculosis. The formation of GSNO is thought to increase the availability of NO, across a wider area and, thus, to play a significant role in the death of pathogenic organisms. At the same time, M. tuberculosis cells have been shown to be sensitive, that is subject to cell death, after exposure to glutathione alone (Venketaraman). Thus, glutathione plays a significant role in the control of infection by intracellular pathogens such as M. tuberculosis and other mycobateria. This effect is due to the increased availability of glutathione for its direct cidal effect as well as the protection against RNS release and the formation of GSNO generated during oxidation stress. Thus, glutathione plays a role directly and indirectly in the antimicrobial activity of immune cells, especially macrophage cells. At the same time, the production of Nitric Oxide synthase has been shown to be dependent on adequate availability of reduced glutathione (Hothersall). This apparently is a host defense mechanism to prevent the suicide of the cell by the production of these toxins. The RNS toxins produced are capable of killing not only the invading organism, but also the host cell. This is particularly true if a deficiency of glutathione occurs. Nitric oxide related molecules that are produced in macrophage defense include nitric oxide intermediates and peroxynitrate (ONOO⁻). These materials are toxic as they interfere with several pathways common to cell function such as Glyceraldehyde 3-Phosphate Dehydrogenase (GAPDH), which is an important enzyme involved in the glycolysis and gluconeogenesis pathways. Functions related to GAPDH include mRNA regulation, DNA repair and replication as well as neuronal apoptosis. The NO defense effect also blocks oxidative phosphorylation, the mechanism of cell energy production in the cell.

Macrophages normally maintain a high intracellular level of glutathione, which reflects their functional exposure to high levels of oxidants (Hothersall). This is needed as they are continuously exposed to high levels to superoxide and peroxide produced by infiltrating neutrophils during an inflammatory response. During activation the rate of glutathione recycling whether from resynthesis or re-reduction will increase from 2 hours to 12 minutes. Thus, the importance of glutathione availability in maintaining cell integrity becomes very clear. An object of the present invention is the use of liposomal glutathione to increase the available glutathione in macrophages involved in chronic infection such as HHV-6. Viruses such as HHV-6, as well as HIV are known to increase the production of RNS, as well as other cytokines such as TNF-α.

Focusing on HIV+ patients, and in particular research involving tuberculosis in HIV+ cells, recent research show the surprising and extremely effective results of the invention. The as yet unpublished research compared the effects of liposomal glutathione according to the invention in cells from HIV+ patients which cells were infected by tuberculosis, with NAC. The results show remarkable and surprising results compared with results of adding NAC to cells from HIV+ patients infected with tuberculosis.

The most salient demonstration of these surprising and unexpected results for purposes of this invention and its claims has been in the area of the very difficult combination of HIV+cells which are infected by tuberculosis. This has been shown in vitro and in vivo in an as yet unpublished study: “Glutathione Supplementation Improves Immune Function in HIV+Macrophages,” Morris D, Guerra C, Khurasany M, Guilford T, Venketaraman V, (unpublished, Western University of Health Sciences, Pomona, C A 91766, USA) (“Morris D”).

What is shown by the Morris D et al article is that the liposomal reduced glutathione from Your Energy Systems, LLC has a surprising effect. The liposomal reduced glutathione, was an 8.25% liposomal formulation of reduced glutathione, formulated in accord with the pending application. See Declaration of F. Timothy Guilford, M.D. Even in very low dosages, it shows substantially improved macrophage function in HIV+ individuals against tuberculosis.

While positive effects were noted for both NAC and the liposomal formulation of glutathione (lGSH), unlike mere NAC, lGSH had two unusual effects:

“Treatment of both HIV+ and HIV− macrophages with lGSH resulted in increased levels of reduced GSH (FIG. 1). While these increases were not as large as those observed with NAC treatment, the doses of lGSH (5 and 10 μM) were much lower than the dosage of NAC given (10 mM). This indicates that lGSH is a much more potent method for raising intracellular GSH concentrations.” See Morris et al at p. 13.
The data thus shows an increased effectiveness by a factor of 1000 of lGSH over NAC (the ratio of 10 mM to 10 μM).
“In addition to increasing total and reduced GSH concentrations, NAC and lGSH were able to raise the ratio of reduced GSH to GSSG, indicating that the observed increases in intracellular GSH are due to increased levels of reduced GSH which has functional antioxidant activity (FIG. 1-2). Similar to the observed overall increases in both total and reduced GSH, the improvements in reduced GSH to GSSG ratios were observed to be greater with NAC treatment in comparison to lGSH treatment; however, lGSH was effective at much lower concentrations. Further, lGSH seems to be more effective at replenishing intracellular GSH in HIV+ macrophages than in HIV−macrophages.”
The Morris D paper later explains this phenomenon:
“In a previous study we observed elevated levels of TGF-β in both the plasma and macrophage culture supernatants of HIV+ macrophages [42]. This elevated TGF-β will compromise the amount of GCLC present inside the cell; consequently, supplementing the raw materials [such as with NAC] for de novo synthesis in HIV+ individuals who are over expressing TGF-β will not result in the same increased production of reduced GSH that is observed in individuals who are not over expressing TGF-β. In addition, this phenomenon may explain why lGSH at lower concentrations than NAC is more effective at raising the concentration of reduced GSH in HIV+ macrophages than in HIV−macrophages. Supplementing with an lGSH formulation provides complete GSH molecules to cells, circumventing the enzymatic pathway responsible for GSH production, without the requirement for the cell to construct the tripeptide [43]. This may also explain why treatment with lGSH seems to raise the ratio of reduced GSH to GSSG at much lower concentrations than NAC, as cells treated with NAC will have to produce new molecules of reduced GSH utilizing their own enzymatic machinery. [emphasis added].” Morris et al at pp. 17-18.
“Taken together, the data we have gathered demonstrates a pattern of chronic inflammation brought on by HIV infection which depletes reduced GSH, and impairs the intracellular killing of M. tuberculosis in macrophages (FIG. 6). By supplementing reduced GSH we were able to mitigate the production of reactive oxygen intermediates and improve the ability of macrophages to kill M. tuberculosis intracellularly. Supplementation of GSH also corresponded with increased production of GCLC and decreased production of GSR, further indicators that conditions of oxidative stress are mitigated through the administration GSH supplements. This data makes a compelling argument for the efficacy of GSH supplements in addition to the ART regimen prescribed to HIV+ patients in improving immune function . . . . Even with all these complicating factors, our data indicates that supplementing GSH in combination with anti-retroviral treatment may provide beneficial effects to the host immune system over anti-retroviral treatment alone. As liposomal formulations of GSH provide complete GSH molecules, bypassing the cellular machinery for GSH production, liposomal formulations of GSH may prove to be more effective in supplementing GSH in HIV patients due to the effects of TGF-β production associated with HIV infection, as well as the observed effect at concentrations lower than NAC.” Morris D at 21-22.
In sum, the oral liposomal reduced glutathione that increases intracellular glutathione as shown by the results of the Morris D et al study illustrates the surprising insight of the invention and the unique and unexpected result. It is 1000× more effective than NAC and because it bypasses cell synthesis biochemistry, the liposomal reduced glutathione is more effective to protect tuberculosis infected HIV patients and is an important supplement for anti-retroviral therapy for HIV+ patients.

Another demonstration of surprising effect was in a published article, Levitskaia T et al, “Aminothiol Receptors for Decorporation of Intravenously Administered Co⁶⁰ in the Rat,” 98(1) Health Physics 53-60 (Pacific Northwest National Laboratory (NIH-Public Access Jan. 1, 2011) PMID:19959951. This article showed that although the oral liposomal reduced glutathione was not quite as effective as the intravenous, it was much more effective than plain oral glutathione. On page 58 of the Levitskaia publication, the Co60 levels were reduced by 12 to 43% compared to oral administration of non-formulated glutathione. Or put another way, there was an improvement from 88% to 100% (which is 13.6% improvement) up to an improvement from 43% to 100% (which is 232.6% improvement). And compared with the control group of non-treated animals, there was a tremendous reduction in radioactive agent. Table 3 shows these results. The first column compared to the second column shows the significantly greater reductions in clearance of Co60 by reason of the liposomal reduced glutathione compared to regular non-formulated glutathione.

On p. 58 of the Levitskaia discussion, in the Discussion (2nd col.), there is a reference to the research stating “Unmodified GSH has limited systemic availability and exhibits little functional effect in terms of decorporation; liposomal GSH appears to have the functional capacity to remove 60-Co. [emphasis added].”

Further, in an article by Zeevalk G, Bernard, L, and Guilford, F. T., “Liposomal-glutathione Provides Maintenance of Intracellular Glutathione and Neuroprotection in Mesencephalic Neuronal Cells,” 35(10) Neurochemical Research 1575-87, October 2010 (published on-line, Springer Science+Business Media, LLC, Jun. 10, 2010) PMID: 20535554 (Zeevalk et al), liposomal reduced glutathione was provided by Your Energy Systems of Palo Alto Calif. which is Dr. Guilford's company.

The “Discussion” section in Zeevalk et al at page 1583, summarizes the results: “Two major findings result from these studies; firstly that liposomal-GSH can be utilized for repletion and maintenance of intracellular GSH in neuronal cells and secondly, that liposomal-GSH can provide significant protection to neurons in a model system relevant to Parkinson's disease.”

“Other approaches such as the use of cysteine, NAC and ethyl esters of glutathione while effective in repleting and in the case of the ethyl ester, elevating intracellular GSH, have limited usefulness due to potential toxicities [14, 17-19, 21]. [emphasis added].”
“Facilitation of intracellular GSH repletion was greatly enhanced by liposomal delivery. The concentration needed for half maximal repletion in mixed mesencephalic cultures containing approximately 70% neurons and 30% glia (unpublished observations) was 100-fold less when GSH was encapsulated into liposomal vesicles (4.75 μM for liposomal-GSH versus 533 μM for non-liposomal fully reduced GSH).” Zeevalk at 1583.
“In summary, the studies presented here show that a liposomal preparation of GSH is 100-fold more potent than non-liposomal-GSH in providing substrates for maintenance of intracellular glutathione in neuronal cells and provides complete protection of neurons in an environmental model of Parkinson's disease. Zeevalk at 1585.

Other surprising results can be seen from the attached article: Rosenblat M, Volkova N, Coleman R, Aviram M., “Anti-oxidant and anti-atherogenic properties of liposomal glutathione: studies in vitro, and in the atherosclerotic apolipoprotein E-deficient mice,” Atherosclerosis. 2007 December; 195(2):e61-8. Epub 2007 Jun. 22 (Abstract). PMID 17588583 (Rosenblatt et al):

The Abstract points out that:
“Liposomal glutathione, but not the control liposomes (with no glutathione), dose-dependently inhibited copper ion-induced low density lipoprotein (LDL) and HDL oxidation . . . . Analyses of cellular cholesterol fluxes revealed that, liposomal glutathione (12.5 mg/kg/day) consumption, decreased the extent of oxidized-LDL (Ox-LDL) uptake by 17% and the cellular cholesterol biosynthesis rate, by 34%, and stimulated HDL-induced macrophage cholesterol efflux, by 19%. Most important, a significant reduction in macrophage cholesterol mass (by 24%), and in the atherosclerotic lesion area (by 30%) was noted. We thus conclude that liposomal glutathione possesses anti-oxidative and anti-atherogenic properties towards lipoproteins and macrophages, leading to attenuation of atherosclerosis development.”

The invention, by achieving a high concentration of reduced glutathione, exhibits unusual and surprising results not suggested or anticipated by the prior literature in treating tuberculosis. No other literature teaches GSH, much less liposomally formulated GSH at 8.25% (Off. Act. 10/20/11 at 15) including for treating TB.

The liposomal glutathione formulation in the doses contemplated herein can be useful to remove increased cobalt and other metallic contaminants from patient's blood due to trace metals leeching from metal prostheses used in hip and knee replacement surgeries.

T lymphocytes have a weak cysteine transporting activity and are consequently unable to increase the level of intracellular glutathione at a high rate, particularly in the inflammatory microenvironment that occurs with T cell activation (Droge, 1991). Antigen presenting macrophages, on the other hand, are known to have a relatively high cysteine transport activity. These macrophages can shift the T cells in their location from a prooxidant state to an antioxidant state, which is an important component of regulating inflammation. When T cells do not get sufficient cysteine, or from the macrophage, their level of glutathione drops, and DNA synthesis decreases (Droge, 1991). This can leave the cells in a state of unregulated inflammation and forms the basis of what is called the shift from the more efficient form of immune function, TH1 to TH2, the state associated with chronic inflammation (Peterson). Restoring the levels of glutathione to macrophages and lymphocytes trapped in the cycle of chronic inflammation restores cells to TH1 function. This effect seems to occur rapidly with the present invention and is one of the probable mechanisms associated with the rapid clinical improvement recounted in clinical Example Case 1.

The HHV-6 virus can trigger recognition responses from the γδ T cell, with resulting reaction to and killing of cells that contain HHV-6 infection. However, HHV-6 has been shown to also infect the γδ T cells, causing them to lose their effectiveness and to die within days of the infection (Lusso). This may represent a strategy that allows for HHV-6 virus to escape immune detection.

It turns out that the HHV-6 virus plays an even larger role in immune suppression. While it has been demonstrated that the HHV-6 virus has been associated with T-cells, its original name, Human B cell lymphotrophic virus may have been valid also. In fact, the HHV-6 may be more aptly named the human immunotrophic virus as it has been shown to infect several critical components of the human immune system such as macrophages, dendritic cells, fibroblasts epithelial cells and bone marrow progenitors. Because of its spectrum of infective action, HHV-6 may have a broad immunosuppressive activity (Lusso).

The cell receptor for HHV-6 is a common type-1 glycoprotein that is a member of the complement activation family Complement is a family of proteins that are involved in destroying cells. This occurs by the conversion of the inactive form of complement to an active, enzymatic form capable of killing both invaders and normal cells. Certain proteins will inactivate this action and prevent complement from damaging normal cells, and this is one of the function of the cluster of proteins known as CD46. CD46 is a membrane protein which known to bind and inactivate complement C3b and C4b (Cattaneo). The binding and inactivating of C3b and C4b protects human cells from lysis by autologous complement. The CD46 cell marker has been found on the immune activator and modulating cells called dendritic cells and macrophages as well as astrocytes in the brain. CD46 is present to some degree on the surface of all nucleated cells (De Bolle). The CD46 is not only a marker, as it has been found to also provide both a site for attachment for certain pathogens like HHV-6 and also allows for a portal of entry of this virus into the cells. Measles virus (MV) is another viral pathogen that uses CD46 for entry into cells. Both MV and HHV-6 have similar capacity to have a negative affect on immune function through attachment to CD46 of immune cells (Kurita). CD46 has been shown to be a receptor for several human pathogens: an enveloped RNA virus (measles virus [MV]), an enveloped DNA virus (human herpesvirus 6), a non-enveloped DNA virus (adenovirus of different serotypes), and two types of bacteria (Streptococcus pyogenes and pathogenic Neisseria) (Cattaneo

It has also been shown that CD46 not only regulates complement function but fine tunes the T-cell-mediated cellular response, bridging the areas of immune defense known as innate and acquired immunity (Cattaneo. While Pathogen binding to the CD46 receptor has been shown to both up-regulate, that is increase immune function and decreased immune function or down-regulation of immune function. The immune suppression mechanism is related to an interference with intracellular immune response at a minimum (Cattaneo) and can include suppression of TH1 function. Thus viral attachment and entry into cells through CD46 can have both direct immune suppression effects and indirect. The indirect effect occurs when the cell function is increased and there is an increased demand for glutathione, creating the situation of glutathione deficiency and a switch to TH2 function.

Gene array analysis of cells that have been infected with HHV-6 demonstrate that the infection can induce pro-inflammatory and decrease anti-inflammatory gene expression. T cell lines that support the replication of both HHV-6 type A and Type B virus called Sup T1 cell lines are used to evaluate the response of T cells to infection with HHV-6. Infection with HHV-6 shows that several genes associated with the immune response are up-regulated, that is determined by an increased mRNA response. The genes that are upregulated include those for IL-18, IL-12 receptor, tumor necrosis factor (TNF) receptor superfamily members and associated signaling molecules including TRAF3 and CD4 (Mayne).

OK ran out of steam here

There are also differences in the expression of different inflammatory mediators that are released from T cells depending on which type of HHV-6 virus type A or Type B they are infected with. Compared to HHV-6A infected cells, HHV-6B infected T cells had elevated levels of several pro-inflammatory molecules, including TNFα and lymphotoxin receptor family and others (Mayne).). Lymphotoxins are biochemicals released by killer T cells. These cells develop from monocytes exposed to IL-2, and are part of the specific immune system that look for and destroys abnormal cells such as tumor cells. An increase in the receptors for these lymphotoxins increases the effect of the toxin, and increases the stress on the cell which is displaying these receptors. A receptor is like a lock and if there is an increase in the number of receptors, there will tend to be more activation of those locks by a chemical key called a ligand, in this case the lymphotoxin. Therefore, ligands act as a key to the receptor and if there are increased receptors, there will be more activation in a cell of whatever effect the receptor can have on the cell. In this situation, the increase in the receptors for lymphotoxin are being displayed in normal cells that have been invaded by virus, causing an increased susceptibility to the toxins. As biochemical toxins increase the oxidation stress on a cell, this causes an increased need for glutathione in the affected cell, and due to the accompanying cascade of free radicals that accompanies the destruction of a cell, in the cells around the affected cell. This effect multiplies as more cells, even uninfected cells are affected by the oxidative damage leading to the symptoms associated with the virus. In addition, additional deleterious effects are associated with infection by HHV-6 Type A cells, in turn had other inflammatory genes up-regulated including the genes for synthesis of phospholipase D2, NF-kB inducing kinase, and nitrogen oxide synthase. By up-regulation, we mean not that more genes are created, but that the activity of a gene is increased as a result of stimulation as the cell encounters certain biochemicals or changes in the environment of the cell.

Additional impact on inflammation was observed in the supT1 cell line infected with HHV-6 with down-regulation of IL-10 protein, an anti-inflammatory cytokine found to be formed by the T cells. The IL-10 cytokine is associated with promotion of TH1 response, lessening the effects of chronic inflammation. IL-10 formation was decreased by infection with both HHV-6A and HHV-6B, suggesting that both types have a stimulus toward down-regulating the TH1 response of T cells (Mayne). This response will cause a further increase in the chronic inflammation response after HHV-6 infection. Thus infection with HHV_{—}6 of either type can lead to compromise of T cell function by a mechanism that can be reversed with the present invention. It is an object of the present invention to reduce the immune suppression that can accompany viral infections such as HHV-6. The coordinated interaction of the both of the TH1 and TH2 systems leads to the efficient removal of viral invaders. When the efficiency of the Th1 system is decreased and the TH2 system is correspondingly increased the effect is a continued release of inflammatory mediators. When this response causes tissue damage it is referred to as chronic inflammation. Thus, the term “chronic”, while generally connoting the passage of time can also occur in the short period of time associated with the onset of a virus if the balance between the two categories of immune response is uncontrolled. The ability to aid the correction of this loss of balance and coordination that occurs during inflammatory reactions and results in tissue damage is the focus of the present invention. The use of the liposomal encapsulated reduced glutathione allows the rapid return of control to a system that has been “cascading out of control”.

A series of unusual events occurs with HHV-6 infection that seems to increase its damaging effects. In cell culture it has been shown that T cells that were not initially carrying the CD4 marker will adopt this marker. This transition to CD4+ will even occur in cells that a near infected cells, but not infected themselves. It appears that this occurs both from an increased amount of this protein cluster being formed and released into the microenvironment. It is also thought that the production of the cytokine IL-18 that occurs with HHV-6 infection will increase the formation of CD4+ cells in the environment adjacent to cells infected with HHV (Akira). The increased release of IL-18 could affect the cells in the local cellular environment during HHV-6 replication, and combined with the increase production of the proteins associated with CD4 account for the increased production of CD4+ that occurs during HHV-6 infection. This transformation is particularly important if there is a co-infection with the HIV virus. The killing of adjacent cells that are apparently not infected by HHV-6 is likely to be due to a combination of these different free radical cascades. A similar event has been demonstrated to occur in the natural control of abnormal cells such as tumor cells and is called intercellular induction of apoptosis (Bauer). In the case of the intercellular induction of cell death observed to occur with HHV-6 infection of T cell cultures the confluence of the formation of ROS and RNS is likely to be at play.

HHV-6 appears to play a significant role in the pathology of Acquired Immuno-Deficiency Syndrome (AIDS), in which loss of CD4 cells is a primary marker. Amongst Peripheral blood mononuclear cells (lymphocytes), CD4 cells are the major targets for HHV-6 infection. Upon entry into CD4 cells the HHV-6 replication process takes several days to initiate, with cytopathic, or cell killing effects becoming visible in 3 to 5 days after infection. The virus requires activation of the CD4 cells in vivo for replication. The changes observed include cell membrane blebbing (small bubbles which appear on the surface of the cell, swelling and induction of multinucleated cells all of which together is called syncytia. The ultimate process of cell death of the CD4 cells is via apoptosis, which is where a cell chemically signals its own death, as opposed to cell necrosis which is caused by an external effect, such as being crushed or poisoned. Of significance is the observation that the cell death phenomenon of apoptosis seems to also involve virus-negative bystander cells (Inoue). It is note worthy that the degree of DNA fragmentation from infected cells in cell culture increased when HHV-6 inoculated cells were cultured in the presence of Tumor Necrosis Factor alpha (TNF-α). These findings were observed in both the subtypes of HHV-6, types A and B, and point out the increased damage that occurs to cells in the presence of TNF-α. It is possible that the combination of increased TNF-α release and the migration of NO out of cells where it is being formed in excess after the HHV-6 infection could account for the death of adjacent cells that do not have viral infection in the cell culture. The effect of the increase in sensitivity to oxidation stress, combined with the influx of NO leading to the formation of RNS free radicals may cause enough cell damage to induce apoptosis.

The attraction to a specific cell or tissue type, known as trophism, of HHV-6 is quite broad. It has been shown to infect lymph nodes, lymphocytes, macrophages, monocytes, kidney tubular epithelium, salivary glands, and tissues of the central nervous system, such as neurons, oligodendrocytes (Braun). HHV-6 has also been found in lungs, genital tract, and brain tissues such as astrocytes, microglia. The infection of brain tissue is through the CD46 receptor, which been demonstrated on various neural cells (Santoro, Soldan). Thus, the attraction of HHV-6 to the CD46 receptor can lead to systemic infections.

Receptors are like a chemical lock, which when triggered by the appropriate biochemical key called a ligand will initiate a response from the cell. These biochemical keys are also known as signaling molecules may trigger a variety of responses ranging from change in the cell metabolism, changes in membrane potential or in the situation of the CD receptors we are reviewing a change in gene expression. The rate at which a signal influences change will depend on both the number of the signal molecules and the number of receptors available. As the number of receptors increases, the opportunity to respond increases and, of course, the reverse occurs if the receptor sites are blocked.

The action of a virus is to increase its number and this is known as replication. Both HHV-6A and HHV-6B can be replicated in cell culture, with activated primary T cells. Some isolates have been adapted to grow efficiently in continuous T-cell lines including the GS strain of HHV-6A, which replicates in HSB-2 cell lines (Braun). HHV-6B is grown most often in primary lymphocytes such as the Molt-3-T cell line. HHV-6 has been propagated in cell lines of other tissues such as neural (nervous system), epithelial (skin), and fibroblastic. The cell line used in the example “LIPOSOMAL GLUTATHIONE ANTIVIRAL EFFECT ON HHV-6 INFECTED CELL CULTURE” is from the T cell line known as HSB-2.

In tissue culture studies HHV-6 It has been shown that the co-infection of the individual CD4 cells with HHV-6 and HIV will accelerate the speed of HIV expression and cell death related to HIV (Lusso). In addition, infection with HHV-6 has been shown to induce reorganization of the expression of cell markers such that cells that normally do not carry the CD4 marker, so called CD4−, such as NK cells, CD8+ T cells and lymphomyeloid progenitor cells are stimulated to begin expressing CD4 markers. This reorganization of expression makes these cell lines more vulnerable to the HHV-6 virus and ultimately cell death by the mechanisms described. It is an object of the present invention that the use of liposomal glutathione will reduce the immunosuppressive effect and delay the progression of immune deficiency in individuals infected with simultaneous infections of HIV and HHV-6. In addition, as deficiency of glutathione is associated with impaired survival in HIV disease (Herzenberg), it is an object of the invention that the use of liposomal glutathione will increase survival of individuals infected with HIV.

HHV-6 infection of white blood cells leads to an increased expression of IFN-α, CD4, and tumor necrosis factor α (TNF-α). The induction of CD4 in lymphocytes that did not previously express CD4 markers rendered these previously refractory lymphocytes susceptible to infection with HIV-1 (Folks). Increases in TNF-α are also known to enhance human immunodeficiency virus type 1 expression (Flamand). This effect may have pathogenic implications in the progression of HIV disease (Braun). At the same time, the up-regulation of TNF-α, and increased production and release of TNF-α may add a stress onto both infected cells and surrounding cells that makes them more vulnerable the effects of oxidative stress. This series of events causes an increased effect of immune suppression in HIV patients and may result in a progression disease in HIV positive individuals who also acquire HHV-6 infection. The use of the present invention should slow the progression of illness in individuals with coinfections with HIV and HHV-6.

The net effect of infection with HHV-6 in cell cultures is a continuation of protein synthesis at levels even greater than those seen in freshly stimulated lymphocytes, accompanied by an inhibition of cell division. Thus the linkage with cytoplasmic growth and cell division appears to have been disrupted, resulting in the common observation of cell cultures infected with HHV-6 of the production of large cells. This continuation of host cell protein synthesis may include the increased production of cytokines such as TNF-α.

Increases in TNF-α are known to occur in many situations associated with viral infection. While it is an integral part in the protection against viral and bacterial infection, the release of TNF-α can result in deleterious effects in certain situations. For example, increased TNF-α release has been shown to enhance human immunodeficiency virus type 1 expression (Flamand).

TNF-α can create a situation that causes a devastating effect on cells deficient of glutathione. This occurs as TNF-α both requires the increased production of glutathione and at the same time increases the need for more glutathione to be produced. TNF-α factor is an inflammatory cytokine that causes damage by generation of oxidative stress. TNF-α has been shown to sensitize cells to injury from peroxide (H₂O₂). Peroxide is an oxidant produced by various cells responding to viral infection including polymorphonuclear cells, natural killer (NK) cells and T-killer cells. The presence of TNF-α even in low concentrations increases the permeability of cells, such as the endothelial cells lining the respiratory tract, to damage from H₂O₂ peroxidation. The amount of reduced glutathione contained in cells has been shown to be decreased in a concentration-dependent fashion upon exposure to TNF-α.

It appears that TNF-α decreases the availability of reduced glutathione, resulting in an increase in local oxidation stress, and at the same time sensitizes the membrane of the cell to increased damage from oxidation stress. The formation of the oxidized form of glutathione, GSSG, can accumulate when its rate of formation exceeds the cells ability to convert it back to reduced glutathione, GSH. In this situation, GSSG can be extruded out of the cell into the extracellular space, or can form mixed disulfides with intra or extracellular proteins resulting in a net loss of total glutathione inside the affected cell (Ishii).

The resulting deficiency of glutathione leaves normal cells exposed to TNF-α induced peroxidation damage. Thus, the normal response of the immune system, in the presence of a glutathione deficiency, in fact worsens the symptomatic condition because the membrane of the normal cells becomes more susceptible to peroxidation damage. Peroxidation damage directed at diseased cells or infectious agents is a desired response; however, such damage directed at normal cells is undesirable.

When normal cells begin to suffer the oxidation stress, the negative effects of TNF-α peroxidation and the reduction in cell glutathione can reinforce each other to the detriment of any cell. First, the release from the immune and epithelial cells of TNF-α is unregulated, and second, cells become progressively more sensitive to peroxidation damage as a result of continued TNF-α release, exacerbating local oxidative stress, which goes on to lessen available glutathione, often resulting in intensification of symptoms. This situation is may be present with any viral infection, and is compounded with in the HHV-6 infection because of the increased formation of NO and RNS placing increased demands on the glutathione system.

Diseases associated with HHV-6 are difficult to isolate as it is difficult to ascertain if the infection is the caused due to HHV-6 or if the HHV-6 has become activated from the latent state by the stress of the initial infection.

Chronic fatigue syndrome has been associated with HHV-6, but it has not been determine definitively that it is caused by HHV-6. Chronic fatigue syndrome (CFS) is not well understood and is difficult to diagnose. CFS is characterized by debilitating fatigue that lasts more that 6 months and does not resolve with bed rest. CFS is accompanied by a variety of symptoms including fever, sore throat, myalgia, lymphadenopathy, sleep disturbances, neurocognitive difficulties, and depression (Holmes). The onset of symptoms often follows a flu-like illness. As the symptoms seem to follow a viral infection like onset, have persistent symptoms of viral like infection an have been reported to have elevated viral antibody levels in test results, a viral origin is suspected. HHV-6 has the characteristics that are most associated with this illness (Wallace).

Blood studies using Enzyme Immuno Assays (EIA) for the detection of IgG and IgM antibodies to HHV-6 early antigen have demonstrated increased rates of serologic elevation in individuals with symptoms of CFS. The early antigen elevation is thought to be characteristic of viral infection and has been demonstrated to be diagnostic of early infection with other Herpes family viruses such as EBV (Patnaik). However, another study using evaluation for DNA of the HHV-6 virus in individuals with clinical symptoms of CFS has failed to confirm the presence of the virus (Wallace).

There is growing concern that infection with the organism borrelia Bergdorferi, also known as Lyme disease can lead to symptoms that are very similar to chronic fatigue syndrome. This is thought to involve a central nervous system or brain infection with the bacteria, and is termed Lyme neuroborreliosis. The mechanism of the damage to nerve cells has not been completely documented, but there is evidence of increased production of cytokines such as TNF-α and may be a mechanism of the neural damage (Garcia). As the central nervous system (CNS) fluid of individuals with Lyme related encephalitis (Pancewicz, 2002). In addition a shift toward the TH2 response has been documented in Lyme disease afflicted individuals (Dattwyler). In addition there is increased oxidation stress in individuals with Lyme disease (Pancewicz, 2001). The inflammatory and oxidation responses that accompany Lyme disease related infection and particularly encephalitis is similar to that seen with HHV-6. The mechanism is probably typical of all forms of encephalitis. An object of the present invention is the use of liposomal encapsulated reduced glutathione for management of lyme disease particularly the neurologic manifestation of the disease known as Lyme neuroborreliosis. The present invention may also be used in conjunction with antibiotic therapy that is oriented toward killing the bacterial organism itself. The choices may include a wide list of antibiotics such as tetracyclines, and penicillin related antibiotics.
The present invention may be combined with antibiotics as well. These antibiotics would include, but not be limited to:
1. Aminoglycoside==Gentamicin, Tobramycin, Netilmicin, Amikacin, Streptomycin.
2. Cephalosporins=Cefazolin, Cefuroxime, Cefotetan, Ceftriaxone, Ceftazidine.
3. Clindamycin
4. Macrolides=Erythromycin, Clarithromycin, Azithromycin.
5. Metronidazole
6. Penicillins such as Penicillin, Ampicillin, Nafcillin, Piperacillin. With or without Aztreonam, Imipenem, or with Beta-lactamase inhibitor including, Ampicillin/sulbactam (Augmentum) or Pipercillin/tazobactam and Beta-lactam=Ceftriaxone, Cefuroxime
7. Quinolones=Ciprofloxacin, Ofloxacin, Gatifloxacin or Trovafloxacin
8. Tetracyclines=Tetracycline, Doxycycline, or Minocycline
9. Trimethoprim-Sulfamethoxazole
10. Vancomycin
11. Chloramphenicol
12. Erythromycin
13. telithromycin a ketolide antibiotic
   Some of these pharmaceutical substances just listed can serve as anti-tuberculosis drugs. Additional tuberculosis drugs include:

### First-Choice Medicines:

| Generic Name | Brand Name |
|---|---|
| ethambutol | Myambutol |
| isoniazid | |
| pyrazinamide | |
| rifabutin | Mycobutin |
| rifampin | Rifadin, Rimactane |
| rifapentine | Priftin |

### Second-Choice Medicines:

| Generic Name | Brand Name |
|---|---|
| amikacin | |
| capreomycin | Capastat Sulfate |
| cycloserine | Seromycin |
| ethionamide | Trecator |
| levofloxacin | Levaquin |
| moxifloxacin | Avelox |
| para-aminosalicylic acid | Paser |
| streptomycin | |

### Combination Medicines:

| Generic Name | Brand Name |
|---|---|
| isoniazid plus pyrazinamide plus rifampin | Rifater |
| isoniazid plus rifampin | Rifamate |

### Dose Example of Anti-Tuberculosis Therapy

The standard treatment is to take isoniazid (INH)—5 mg/kg/day (max 300 mg daily; rifampin—mg/kg PO qD; ethambutol (EMB)—15 mg/kg orally once a day; and pyrazinamide (PZA) Daily dosing:
40 to 45 kg: 1000 mg
56 to 75 kg: 1500 mg
76 to 90 kg: 2000 mg
for 2 months. Treatment is then continued for at least 4 months with fewer medicines. Such as INH 5 mg/kg/day (max 300 mg daily; rifampin—10 mg/kg PO qD

### Combined With

Liposomal glutathione 1.5 teaspoons (7.5 cc) orally twice a day.
Dose for HIV and TB: The dose for treatment of an individuals with HIV and TB is the same as for an individual with TB alone with the ARV drugs taken simultaneously.

### Suggested Dosing for Anti-Tuberculosis Drugs

This information is compiled and taken from the Centers for Disease Control website from among the reports at Morbidity and Mortality Weekly Report, Centers for Disease Control and Prevention, 1600 Clifton Rd, MailStop E-90, Atlanta, Ga. 30333, U.S.A. It is based on an article published at the American Journal of Respiratory and Critical Care Medicine (vol. 167, pages 603-62)(2003).
These are alternative preferred modes of invention for combination with administration with liposomal reduced glutathione formulated according to the invention.
For culture—positive pulmonary tuberculosis caused by drug-susceptible organisms, there are four preferred options with various continuation phases

| Drugs | Interval and doses (minimal duration) |
|---|---|
| a) Initial phase | |
| Isoniazid | 7 days per week for 8 weeks for 56 |
| (INH) or | doses |
| Rifampin | |
| (RIF) or | |
| Pyrazinamide | |
| (PZA) or | |
| Ethambutol (EMB) | |
| Continuation Phase and | |
| follow-up | |
| 1) INH/RIF | Seven days per week for 18 weeks for |
| | 126 doses or 5 days week for 18 weeks |
| | for 90 doses |
| 2) INH/RIF | Twice weekly for 18 weeks for 36 doses |
| 3) INH/RPT | Once weekly for 18 weeks for 18 doses |
| b) Initial phase | |
| Isoniazid | Seven days per week for 2 weeks for 14 |
| (INH) or | doses, then 2 × per week for 6 weeks for |
| Rifampin | 12 doses or 5 days per week for 2 weeks |
| (RIF) or | for 10 doses, then twice weekly for 6 |
| Pyrazinamide | weeks for 112 doses |
| (PZA) or | |
| Ethambutol (EMB) | |
| Continuation Phase and | |
| follow-up | |
| 4) INH/RIF | Twice weekly for 18 weeks for 36 doses |
| 5) INH/RPT | Once weekly for 18 weeks for 18 doses |
| c) Initial phase | |
| Isoniazid | Three times per week for 8 weeks for 24 |
| (INH) or | doses |
| Rifampin | |
| (RIF) or | |
| Pyrazinamide | |
| (PZA) or | |
| Ethambutol (EMB) | |
| Continuation Phase and | |
| follow-up | |
| 6) INH/RIF | Three times per week for 18 weeks for |
| | 54 doses |
| d) Initial phase | |
| Isoniazid | 7 days per week for 8 weeks for 56 |
| (INH) or | doses or 5 days per week for 8 weeks |
| Rifampin | for 40 doses |
| (RIF) or | |
| Ethambutol (EMB) | |
| Continuation Phase and | |
| follow-up | |
| 7) INH/RIF | Seven days per week for 31 weeks for |
| | 217 doses or 5 days week for 31 weeks |
| | for 155 doses |
| 8) INH/RIF | Twice weekly for 31 weeks for 62 doses |

In all of the regimens for culture positive pulmonary tuberculosis caused by drug-susceptible organisms, evidence suggests that five continuous days out of seven would be adequate, but only where treatment on site or in a health care facility is contemplated (more generically referred to as DOT or Directly Observed Therapy). Patients with cavitation on initial chest radiograph and positive cultures at completion of 2 months of therapy should receive a 7-month (31 week; either 217 daily doses or 62 doses (twice weekly)) continuation phase. Continuation Phase Options 3 and 5 should only be used with HIV negative patients who have negative sputum smears at the time of completion of 2 months of therapy and who do not have cavitation on their initial chest radiograph. Continuation Phase Options 3 and 5 are not recommended for HIV+ patients, but may be successful with liposomal reduced glutathione. Patients started on either of these regimens who have a positive culture from the 2-months specimen need extended treatment for an extra 3 months. Single daily doses of ethionamide can be given at the main meal or at bedtime. Rifampin and rifabutin may be adjusted if given to AIDS patients taking protease inhibitors or non-nucleoside reverse transcriptase inhibitors.

### Doses of Anti-Tuberculosis Drugs for Adults and Children

| | | | | |
|---|---|---|---|---|
| Drug/preparation | Daily | 1×/wk | 2×/wk | 3×/wk |

First-line drugs

| | | | | | |
|---|---|---|---|---|---|
| Isoniazid | Adults | 5 mg/kg | 15 mg/kg | 15 mg/kg | 15 mg/kg |
| Tablets (50 mg. | | (max. 300 mg) | (max. 900 mg.) | (max 900 mg) | (900 mg) |
| 100 mg. 300 mg.); | Children | 10-15 mg/kg | Not Applicable | 20-30 mg/kg | N/A |
| elixir (50 mg/ml); | | (max. 300 mg) | (N/A) | (max. 900 mg) | |
| aqueous solution | | | | | |
| (100 mg/ml) for | | | | | |
| intravenous or | | | | | |
| intramuscular | | | | | |
| injection | | | | | |
| Rifampin | Adults | 10 mg/kg | Not Applicable | 10 mg/kg | 10 mg/kg |
| Capsule (150 mg, | | (max. 600 mg) | (N/A) | (max 600 mg) | (600 mg) |
| 300 mg); powder | Children | 10-20 mg/kg | Not Applicable | 10-20 mg/kg | N/A |
| may be suspended for | | (max. 600 mg) | (N/A) | (max. 600 mg) | |
| oral administration; | | | | | |
| aqueous solution | | | | | |
| for intravenous | | | | | |
| injection | | | | | |
| Rifabutin | Adults | 5 mg/kg | Not Applicable | 5 mg/kg | 5 mg/kg |
| Capsule (150 mg) | | (max. 300 mg) | (N/A) | (max. 300 mg) | (max. 300 mg) |
| | Children | Children dose | Children dose | Children dose | Children dose |
| | | unknown | unknown | unknown | unknown |
| Rifapentine | Adults | Not Applicable | 10 mg/kg | Not Applicable | Not Applicable |
| Tablet (150 mg, | | (N/A) | (continuation | (N/A) | (N/A) |
| film coated) | | | phase) (max | | |
| | | | 600 mg) | | |
| | Children | Not approved | Not approved | Not approved | Not approved |
| | | for child use | for child use | for child use | for child use |
| Pyrazinamide | Adults | See Table 4 | Not Applicable | See Table 4 | See Table 4 |
| Tablet (500 mg | | | (N/A) | Data below | Data below |
| scored) | Children | 15-30 mg/kg | Not Applicable | 50 mg/kg | Not Applicable |
| | | (max. 2.0 g) | (N/A) | (max. 2 g) | (N/A) |
| Ethambutol | Adults | See Table 5 | Not Applicable | See Table 5 | See Table 5 |
| Tablet (100 mg, | | | (N/A) | data below | data below |
| 400 mg) | Children | 15-20 mg/kg | Not Applicable | 50 mg/kg | Not Applicable |
| | | daily (max. | (N/A) | (max. 2.5 g) | (N/A) |
| | | 1.0 g) | | | |

Second-line drugs

| | | | | | |
|---|---|---|---|---|---|
| Cycloserine | Adults | 10-15 mg/kg/ | No intermittent | No intermittent | No intermittent |
| Capsule (250 | | day (max 1.0 | administration | administration | administration |
| mg) | | g in two doses), | data available | data available | data available |
| | | usually 500-750 | | | |
| | | mg/day in two | | | |
| | | doses(may not | | | |
| | | be tolerated | | | |
| | | well-testing | | | |
| | | of serum conc. | | | |
| | | may be needed | | | |
| | Children | 10-15 mg/kg/d | Not Applicable | Not Applicable | Not Applicable |
| | | (max. 1.0 g/d) | (N/A) | (N/A) | (N/A) |
| Ethionamide | Adults | 15-20 mg/kg/d | No intermittent | No intermittent | No intermittent |
| Tablet (250 mg) | | (max. 1.0 | administration | administration | administration |
| | | g/d), usually | data available | data available | data available |
| | | 500-750 | | | |
| | | mg/day in a | | | |
| | | single daily | | | |
| | | dose or two | | | |
| | | divided doses | | | |
| | Children | 15-20 mg/kg/d | No intermittent | No intermittent | No intermittent |
| | | (max. 1.0 g/d) | administration | administration | administration |
| | | | data available | data available | data available |
| Moxifloxacin | Adults | 400 mg daily | No intermittent | No intermittent | No intermittent |
| Tablets (400 | | | administration | administration | administration |
| mg); aqueous | | | data available | data available | data available |
| solution (400 | Children | N/A | N/A | N/A | N/A |
| mg/250 ml) for | | | | | |
| intravenous | | | | | |
| injection | | | | | |
| Gatifloxacin | Adults | 400 mg daily | No intermittent | No intermittent | No intermittent |
| Tablets (400 | | | administration | administration | administration |
| mg); aqueous | | | data available | data available | data available |
| solution (200 | Children | N/A | N/A | N/A | N/A |
| mg/20 ml; 400 | | | | | |
| mg/40 ml) for | | | | | |
| intravenous | | | | | |
| injection | | | | | |

The table 4 data reference above refers to the following suggested pyrazinamide (PZA) doses, using whole tablets, for adults weighing 40-90 kilograms. The 90 kg figure is the maximum regardless of weight. The weight is to be based on estimated lean body weight.

| Timing | Wt. 40-55 kg | Wt. 56-75 kg | Wt. 76-90 kg. |
|---|---|---|---|
| Daily | 1000 mg | 1,500 mg | 2,000 mg |
| 3 × per week | 1,500 mg | 2,500 mg | 3,000 mg |
| 2 × per week | 2,000 mg | 3,000 mg | 4,000 mg |

The Table 5 data reference above refers to the following suggested ethambutol (EMB) doses, using whole tablets, for adults weighing 40-90 kilograms. The 90 kg figure is the maximum regardless of weight. The weight is to be based on estimated lean body weight.

| Timing | Wt. 40-55 kg | Wt. 56-75 kg | Wt. 76-90 kg. |
|---|---|---|---|
| Daily | 800 mg | 1,200 mg | 1,600 mg |
| 3 × per week | 1,200 mg | 2,000 mg | 2,400 mg |
| 2 × per week | 2,000 mg | 2,800 mg | 4,000 mg |

Because of the unpublished findings, the invention proposes to also address the problem of drug-resistant tuberculosis, also referred to as Multi-Drug Resistant (MDR) tuberculosis (“MDR TB”) or in the literature as drug-resistant pulmonary tuberculosis.

The invention proposes the use of liposomal reduced glutathione in conjunction with the above dosage regimens to treat multi-drug resistant tuberculosis, There are various gradings of effectiveness of dosage regimens; a key factor related to an MDR follow-on regimen is to examine to what drugs the patient may already have resistance.

MDR TB, at the moment, is generally attacked with a combination of drugs. These include primary agents to which the patient does not appear to have resistant (generally seeming to be selected based on what prior therapy was not successful). Thus if the patent appears to be Isoniazid resistant, Rifampin is given with other drugs, and vice-versa. Those other drugs may be primary agents such as ethambutol, isoniazid, pyrazinamide, but often streptomycin or a fluoroquinolone, particularly ofloxacin, levofloxacin or ciprofloxacin are added in. as an alternative to streptomycin, other aminoglycosides such as amikacin, kanamycin or capriomycin are used. Other alternative agents which may be used, particularly where there seems to be both isoniazid and rifampin resistance, are ethionamide, cycloserine, p-aminosalicylic acid, clarithromycin, amoxicillin-clavulanate and linezolid. Use of these agents by injection is sometimes considered an enhancement.
The preferred method of the invention is to determine what primary agent rifampin or isoniazid to which the patient appears resistant. Then, the agent to which the patient is not resistant is utilized in combination with liposomal reduced glutathione to treat the MDR TB. Ciprofloxacin in base dose can be added. If there is not initial success, then alternative agents from the prior paragraph can be added for administration to attempt to curtail the TB disease. Significantly longer periods, of up to 24 months of treatment may be needed to definitively address MDR TB.

The presence of IgM, that is the acute phase immunoglobulin, antibody to the early antigen of HHV-6 was reported to be found in individuals with multiple sclerosis (MS) in 1997 (Soldan). There have been numerous publications linking the association of HHV-6 with multiple sclerosis subsequently. A 2005 study by Meeuwsen et al suggests reviews the possibility that HHV-6 could play a role in the formation of the Central Nervous System (CNS) diseases such as multiple sclerosis.

Multiple sclerosis is a chronic disease of the central nervous system. The symptoms are diverse as the disease involves the breakdown of the protein coating of nerves called myelin. This breakdown causes an interruption of nerve signals from the brain to the peripheral nerves. Demyelination of nerves is the pathologic hallmark of the disease. Symptoms range from mild to severe. Mild symptoms include problems with vision of dexterity problems, numbness, and tingling sensations. More severe symptoms include partial or complete loss of vision and mobility. One of the mechanisms proposed for the disease is an immune cell recognition of myelin as being a foreign substance This may be due to inflammation induced changes causing the myelin to look foreign to the immune system, or to changes associated with viral infection. Regardless of the cause, subsequent T cell activation initiates an inflammatory reaction toward the nerve, resulting in breakdown of myelin and nerve damage.

In the central nervous system, HHV-6 has been associated with the complications of illnesses, including neuro-inflammation, febrile seizures, and encephalitis/encephalopathy. There is speculation that direct invasion of the virus into the CNS may play an important role in causing these neurologic complications (Yoshikawa)

The presence of HHV-6 infection of both type A and B in a cell culture will reduce the number of CD4+ cells directly, apparently by inducing apoptosis. This process occurs even in cells that are not infected with virus. Apparently, even the ultracentrifuged supernatant of HHV-6 inactivated with UV light irradiation carries a substance that will induce CD4+ cells apoptosis (Dockrell). The effect of apoptosis is increased if TNF is present in cell culture cells. Other CNS diseases including encephalitis, or brain inflammation that may accompany bacterial or viral diseases, seizures and difficulty with memory and concentration have been associated with HHV-6 infection. Macrophage cell lines expressing human CD46 produce higher levels of nitric oxide upon infection with measles virus in the presence of IFN-γ. This response is dependent on the presence of CD46. The immune suppression seen after measles virus, and HHV-6 is thought to be connected to the stimulation through the CD46 receptor in both macrophages and dendritic cells. Measles virus induces transient suppression of host immunity, leading to secondary infections that are a major cause of death in measles patients (Kurita).

The production of increased amounts of nitric oxide by HHV-6 type B and TNF by HHV-6 type A both lead to increased oxidation stress in cells both carrying HHV-6 and in the local environment of HHV-6. The increased oxidation stress may account for the increase in apoptosis seen in cells infected with HHV-6 as well as the cells in the accompanying microenvironment. It is an object of the invention that the use of liposomal encapsulation of reduced glutathione to deliver reduced glutathione will stabilize the oxidatively stressed cells and allow for increased survival at the cell level resulting in a decrease in symptoms experienced during HHV-6 infection. The method of activity is reviewed in the example “LIPOSOMAL GLUTATHIONE ANTIVIRAL EFFECT ON HHV-6 INFECTED CELL CULTURE”

The increased production of nitric oxide (NO) leads to the production of metabolites of NO. Radicals of NO are formed creating .NO, (the dot “.” is used to show a free electron is available that has a high tendency to bond with any other free electron) which reacts immediately with the abundant free radicals of oxygen such as superoxide, .O₂— creating generating cytotoxic peroxynitrite ONOO— (Johansen).). Superoxide not only has lots of free electrons, but is negatively charged and can both ionically bond with a positive charge, as well as bond covalently by sharing its electron with another unshared or free electron. .NO is normally produced from L-arginine by nitric oxide synthase (NOS), which has been noted to be up-regulated in cells infected with virus that accesses cells through CD46, such as measles and HHV-6 type A (Mayne). Superoxide is produced by a large number of normal cell oxidase reactions such cyclooxygenase, NAD(P)H oxidase, xanthine oxidase. A consistent source of superoxide radicals in the cell results from the mitochondrial electron transport chain during the course of normal oxidative phosphorylation, which is essential for generating ATP (the basic energy chemical of our bodies). Superoxide (.O₂—) is dismutated to H₂O₂ (hydrogen peroxide) by manganese superoxide dismutase (Mn-SOD) in the mitochondria and by copper (Cu)-SOD in the cytosol. Normally, removal of H₂O₂ occurs by its conversion to H₂O and O₂ by glutathione peroxidase (GSH-Px) or catalase in the mitochondria and lysosomes, respectively. This is a good and natural function; the normal conversion of superoxide to hydrogen peroxide and then to water and oxygen using glutathione is how our body gets rid of normal cell energy cycle wastes. However, If H₂O₂ is not removed, it can be converted to the highly reactive and cell damaging hydroxyl radical .OH⁻, in the presence of transition elements like iron and copper, in a reaction known as the Fenton reaction.

The production of excess free radicals is termed oxidation stress. Oxidation stress has pathological consequences including damage to proteins, lipids and DNA. Oxidation stress damage begins at the level of the intracellular molecular level such as the superoxide radicals formed from mitochondrial function. If adequate amounts of glutathione to support the enzyme glutathione peroxidase are not present to remove the excess H₂O₂, an increase in formation highly damaging .OH radicals will occur leading to damage from oxidation stress. The .OH radical are most dangerous because they not only have a free electron, but also a negative charge so they will chemically bond with almost any compound, which results in a change of the shape and the function of the biochemical bound to the free radical. The .OH radical is known to be particularly damaging to cell membranes.

Viral infection in general is known to decrease glutathione. For example, animal studies on coxsackie virus in mice shows that decreases in plasma glutathione levels identify are associated with increased loss of cardiac cells during the otherwise benign illness with coxsackie virus. (Kyto). Thus, decreased glutathione levels may increase the tissue toxicity of viral infections.

Many viral infections involve an oxidation insult that results in a marked depletion of extra- and intracellular GSH levels. Examples of viral infections that lower GSH include hepatitis C virus (HCV) (Boya), HIV-1 (Buhl, Garaci, 1997; Kalebic), parainfluenza-1, Sendai virus (Garaci, 1992; Palamara, 1996) and herpes simplex virus-1 (HSV-1) (Palmara, 1995).

It has been demonstrated that supplementation of GSH directly or by increasing the availability of its precursor component of cysteine in a combination called N-acetyl cysteine (NAC) will replenish intracellular stores of glutathione diminished in viral infection and the increased level of glutathione will inhibit viral replication. The inhibition of viral replication by an increase in glutathione has been reported for HIV (Garaci, 1997; Kalebic) and HSV-1 (Nucci, Palamara, 1995) and influenza (Cai).

In spite of the research accomplishments, the difficulty in supplying glutathione has slowed the emergence of an oral glutathione therapeutic for virus. The fact that replenishing intracellular glutathione is a desirable goal is referenced by Vogel. However, replenishing glutathione with an oral therapeutic has proven challenging, and there is no reference to the use of a liposomal encapsulation of reduced glutathione to accomplish this task.

Development of the use of glutathione by intravenous (IV) supplementation has also been undeveloped for several reasons. Intravenous supplementation shows only a very short half life in blood plasma. The administration of the intravenous materials is cumbersome, would require repeated administration and creates a significant expense as well as the small but real, risk related to intravenous infusion.

Most prominently, it has not previously been possible to stabilize reduced glutathione in an aqueous solution for intravenous infusion. The formation of an intravenously stable solution of reduced glutathione has been referenced by the author previously in a provisional application filed by the inventor Guilford, Ser. No. 60/594,324 on 2005-03-29 entitled “Administration Of Glutathione (Reduced) Via Intravenous Or Encapsulated In Liposome For The Amelioration Of Flu-Like Viral Symptoms And Treatment And Prevention Of Virus” which is adopted and incorporated herein by reference. The use of intravenous reduced glutathione is also included in the object of this invention for the treatment of HHV-6 and related viral infections.

The intravenous form of the present invention is particularly useful for use in individuals suffering severe encephalitis as is seen in HHV-6, as well as by other causes, such that they are unable to ingest medicaments orally, and intravenous infusion is necessary.

While supplementation with NAC to raise lymphocyte glutathione levels would seem like an attractive solution, studies have shown that in the presence of virus like HIV, supplementation of NAC fails to increase glutathione in lymphocytes and plasma of patients with virus such as AIDS (Witschi, 1995).

Glutathione in a pure powdered form or “neat” form does not appear to be absorbed, as was documented in a study in which 3 grams of glutathione ingested orally did not show any increase in glutathione in the blood (Witschi, 1992). There are no references documenting benefit of oral “neat” glutathione in humans.

Viral infection also impairs the absorption of glutathione from plasma into cells. The normal absorption route of glutathione into cells requires glutathione to be broken down into component parts that are transported across cell membranes and then reconstituted inside the cells as glutathione. This process is often impaired during viral infections (Vogel).

Glutathione participates in the alleviation of oxidation stress of the all tissues in the body. The brain consumes about 20% of the oxygen utilized by the body but constitutes only 2% of the body weight. Oxidative metabolism of brain cells continuously generates reactive oxygen species at a high rate in brain cells. The detoxification of reactive oxygen species such as superoxide and hydroxyl radicals is an important function of glutathione in the brain as removal of reactive oxygen species is essential for brain function. Reactive oxygen species cause damage by lipid peroxidation of the lipids found in cell membranes, causing DNA strand breaks and alteration of proteins such as enzymes. Because the brain tissue is rich in lipids comprised of unsaturated fatty acids, it may be particularly vulnerable to the effects of oxidation stress. The brain contains only low to moderate levels of antioxidant enzymes such as catalase, superoxide dismutase and glutathione peroxidase compared to other tissues in the body such as kidney or liver (Dringen). Catalase does not detoxify organic hydroperoxides, so the glutathione based peroxidase system is required for this function. In order to maintain a constant intracellular glutathione level the glutathione consumed by release or conjugation with toxins must be replaced. The reduction of oxidants directly also consumes the available reduced glutathione as it is oxidized after reacting nonenzymatically with radicals or as the electron donor for the reduction of peroxides in the reaction catalyzed by glutathione peroxidase. While oxidized glutathione can be regenerated by glutathione reductase, this creates a demand on the production of NADPH, NADPH which is one of the important chemicals in generating the ATP, the energy component of our bodies discussed before. A delay in the production of these intracellular constituents puts a greater demand on the availability of reduced glutathione. In this situation glutathione is no longer available in the affected cells by normal production and an exogenous, that is an outside supply is required.

In the normal situation an increased supply from the circulation may be available, but in a system that is deficient in glutathione, the breakdown in production requires glutathione to be supplied exogenously to individual cells as well as the whole system. The presence of oxidation stress such as occurs with toxins such as lead or mercury will decrease the biochemical cycle called the methionine cycle that produces the integral components that lead to cysteine, an essential component of glutathione. Thus, in situations of oxidation stress, glutathione becomes an “essential” cell constituent, that is, the system requires exogenous supply (James).

Because H₂O₂ is the peroxide generated in the highest quantity in the brain, the protection against H₂O₂ related toxicity is particularly important. Astoglial cells have a higher capacity to detoxify peroxide than neurons. When Astroglial cell protection is lost, neurons become more susceptible to damage and brain dysfunction can occur. Thus, Astroglial cell levels of glutathione are particularly important in protecting neurons against peroxide related toxicity (Dringen).

As astroglial cells are also targets of HHV-6 via the CD46 receptor, events and exposures that lower glutathione levels may trigger reactivation of the virus. The activation of cells by HHV-6 has been shown to increase the cytoplasm activity of the cell, which could, in turn increase the antioxidant demands of the cell on the glutathione system. Returning the intracellular level of both immune cells and astrocytes to normal using the present invention protects brain cells from the damage of the infection and increases the likelihood that the normal cell protective mechanisms can defend against the effects of the virus and limit the viral infection.

Other viruses known to affect brain function include HIV viruses. The mechanism of this action is not clear, but is thought to be due to the infection of macrophages that migrate to the brain and effect inflammatory changes (Hans). In addition HIV-1 infected macrophages have been documented to produce toxins such as glutamate, quinolinic acid and nitric oxide, (Cunningham). The presence of the neurotoxins such as quinolinic acid and nitric oxide can increase the demand on the glutathione system (Cruz-Aguado). Nitric oxide can diffuse out of cells that are producing an excess during HHV-6 infection and can diffuse into adjacent cells that are not infected. The conversion of the nitrous oxide to the reactive nitrous oxide intermediates discussed previously may adversely affect these cells.

Quinolinic acid has been demonstrated to increase cytotoxic lipid peroxidation products that break down the cell wall). The increased generation of these products has been demonstrated to be decreased by reduced glutathione (St'astny).

The production of toxins by cells machinery overtaken by virus increases the oxidation stress and demand for glutathione both locally and through out the system. The effect on brain cells may be even greater due to the high lipid membrane content of this organ, and it is conceivable that a brain localized cascade of free radical oxidation stress develops in the brain tissues leading to an encephalitis situation similar to what happens with the cytokine storm and the production of respiratory distress. This would account for the findings of encephalitis associated with viral infection, both with and without evidence of viral infection in the cerebral spinal fluid that has been observed with both influenza and HHV-6 infections (Sugaya).

In a situation of decreased brain cell availability of glutathione such as cells affected by oxidation stress with or without virus, delivery of reduced glutathione to these cells in a liposome is a preferred form of the invention.

The object of this invention is to provide glutathione encapsulated in liposomes for direct utilization in oxidation stressed cells. These cells may be the immune cells involved with viral infection or the neurons of the brain or other tissues of the body that are stressed during infection with bacteria, virus or due to the presence of toxins of endogenous or exogenous origin.

Documentation for the use of the present invention of liposomal encapsulated reduced glutathione to reach brain cells with reduced glutathione level is found in the use of the invention in individuals with Parkinson's disease. This use of the invention has been reviewed in a provisional application filed by the inventor Guilford, Ser. No. 60/522,785 on Nov. 7, 2004 entitled “Liposomal Formulation for Oral Administration of Glutathione (Reduced)” which is adopted and incorporated herein by reference.

An advantage of the present invention is that the liposome composition used is capable of delivery of the active ingredient, reduced glutathione, directly to cells by the mechanism of cell fusion. Liposomes have been documented to fuse to cells and deliver their content into the cells (Constantinescu). The use of glutathione in liposomes has been previously referenced by Smith in U.S. Pat. No. 6,764,693, however Smith references the use of liposomes that are designed to disrupt upon contact with oxidative environments and release their content into the circulation. The liposomes in the present invention are releasing their content not only into the general circulation, but in the preferred mode of action, into cells such as macrophage and viral laden cells undergoing inflammatory changes. Those cells are not necessarily in oxidative environments, and this invention is intended to have prophylactic effect in favor of normal cells to protect them against impending infection and against the cytokine storm effect.

Smith, U.S. Pat. No. 6,764,693, references the activity of his invention as requiring the use of liposomes containing a combination of glutathione with at least one other antioxidant material to increase intracellular and extra cellular antioxidants. This invention eliminates the necessity of at least one other antioxidant material, as the glutathione containing liposome is self-sufficient to act as the necessary antioxidant. The formation of liposomes capable of maintaining glutathione in the reduced state is a novel component of the present invention. The ability to deliver reduced glutathione to sites of inflammation creates a novel compound.

Demopolis et al in U.S. Pat. No. 6,204,248 references the use of glutathione for the treatment of viral diseases such as HIV and herpes family viruses. However, the patent does not reference the use of liposomes for delivery of reduced glutathione. Another oral form of glutathione referenced by Demopolis requires the combination of glutathione with ascorbic acid, apparently to facilitate the absorption of glutathione. This Demopolis reference is for the encapsulation of glutathione with ascorbic acid, however there is no reference for the encapsulation of a liposome enclosing only reduced glutathione as is proposed in the present invention.

As reviewed in the general background discussion, there are previous references to the use of glutathione to inhibit viral replication. However, there are no references demonstrating the use of liposomal encapsulation of glutathione for the treatment of HHV-6 or HIV, particularly HIV in association with tuberculosis.

Likewise, there are no references for the use of the single active ingredient, reduced glutathione, encapsulated in a liposome for the treatment of systemic diseases such as virus for the modulation of the tissue damaging effects of chronic inflammation or the effects of the damaging effects of the cytokine cascade known as the “cytokine storm” Toxin production, such as by the products of nitric oxide or quinolinic acid by invading organisms or by the defense reaction of immune cells each requires glutathione for removal. Thus, the demands on the availability of glutathione are great in cells undergoing infection.

### BRIEF DESCRIPTION OF THE INVENTION

The invention discloses a method of delivering reduced glutathione to a mammalian system in a vehicle, a liposome, that is suitable for the stabilization of cell systems infected with virus such as HIV, or HHV-6 and for the amelioration of symptoms related to viral infection by stabilizing cells locally and systemically to the affects of cytokines and other toxins released during viral infection, and to address tuberculosis infected patients who are HIV+. Patients having tuberculosis includes those with active symptoms of tuberculosis and those who test positive for tuberculosis.

Dose of liposomal reduced glutathione for individuals with HIV and/or TB is a range of ¼ teaspoon (containing 100 mg reduced glutathione) for every 30 pounds up to 100 pounds of weight. The dose over 100 pounds is a range of 1.0 teaspoon to 3.0 teaspoons twice a day. The preferred dose is 1.5 teaspoons twice a day orally in combination with the HIV drugs and/or the anti-tuberculosis drugs.

### FURTHER DETAILED DESCRIPTION OF THE INVENTION

The invention describes a method effected through a composition for oral, topical mucosal (including nasal) or dermal (skin) administration of a combination of glutathione, reduced, in a liposome.

The preferred embodiment of the invention is the method of oral administration for ingestion of the liposomal encapsulation of reduced glutathione for viral and central nervous system infection.

Another embodiment is the administration of the invention for the treatment of viral infection such as HHV-6 by the administration of the of the liposomal encapsulation of reduced glutathione together with the simultaneous administration with a pharmaceutical agent known to be effective against HHV-6, such as ganciclovir, phosphonoformic acid also known as Foscarnet or cidofovir (Dockrell).

The advantage offered by the combination of glutathione with a suitable pharmacologic agent is the increased survival and reduced toxicity of cells as well as individuals infected with virus such as HHV-6. The reduction in toxicity is achieved by the both the protective action of glutathione and the reduction in the amount of pharmaceutical agent needed to achieve antiviral effect. The effect of the combination of antiviral agents and liposomal glutathione has not been previously referenced.

In all embodiments, the key ingredients in the liquid and spray are the reduced glutathione, the water, the glycerin and the liposomal agent. Other ingredients can be adjusted such as potassium sorbate, polysorbate-20, and optional spoilage retardants or taste enhancers. For any w/w/percentage adding up to more or less than 100%, the amount of deionized water content can be adjusted so the total ingredients add up to 100% w/w.

### DETAILED DESCRIPTION OF THE PREFERRED EMBODIMENTS

### Example 1

### Liposomal Glutathione Drink or Spray 2500 mg Per Ounce

| Ingredient | % w/w |
|---|---|
| Deionized Water | 74.4 |
| Glycerin | 15.00 |
| Lecithin | 1.50 |
| Potassium Sorbate | 0.10 |
| (optional spoilage retardant) | |
| Glutathione (reduced) | 8.25 |

A lipid mixture having components lecithin, and glycerin were commingled in a large volume flask and set aside for compounding.

In a separate beaker, a water mixture having water, glycerin, glutathione were mixed and heated to 50. degree. C.

The water mixture was added to the lipid mixture while vigorously mixing with a high speed, high shear homogenizing mixer at 750-1500 rpm for 30 minutes.

The homogenizer was stopped and the solution was placed on a magnetic stirring plate, covered with parafilm and mixed with a magnetic stir bar until cooled to room temperature. Normally, a spoilage retardant such as potassium sorbate or BHT would be added. The solution would be placed in appropriate dispenser for ingestion as a liquid or administration as a spray.

Analysis of the preparation under an optical light microscope with polarized light at 400× magnification confirmed presence of both multilamellar lipid vesicles (MLV) and unilamellar lipid vesicles.

The preferred embodiment includes the variations of the amount of glutathione to create less concentrated amounts of glutathione. The methods of manufacture described in Keller et al, U.S. Pat. No. 5,891,465, Apr. 6, 1999, are incorporated into this description.

### Example 2

### Glutathione LipoCap Formulation

| Ingredient | Concentration % |
|---|---|
| Sorbitan oleate | 2.0 |
| Glutathione (reduced) | 89.0 |
| Deionized water | 4.0 |
| Potassium sorbate | 0.2 |
| Polysorbate 20 | 2.0 |
| Phospholipon 90 (DPPC) | 2.0 |

Components are commingled and liposomes are made using the injection method (Lasic, D., Liposomes, Elsevier, 88-90, 1993). When liposome mixture cooled down 0.7 ml was drawn into a 1 ml insulin syringe and injected into the open-end of a soft gelatin capsule then sealed with tweezers. Large scale manufacturing methods for filling gel caps, such as the rotary die process, are the preferred method for commercial applications. The liposomal glutathione for this invention is and was made by Biozone, Inc. of Pittsburg, Calif. and sold by Your Energy Systems, Inc. of Palo Alto, Calif.

### Preferred Dosing

The preferred dosing schedule of the invention for the treatment of influenza symptoms is 600 mg (1 and ½ teaspoon) of the invention to be taken at the first onset of symptoms. A dose of 400 mg (1 teaspoon) to 600 mg is to be repeated each hour until symptoms are relieved. Once symptom relief is achieved, the dose is repeated immediately upon the return of symptoms. The anticipated amount to be taken is 1 to 2 ounces in 24 hours. See case examples.

If symptoms recur in the following 24 hours the regimen may be repeated as stated.

1 ounce is 5.56 teaspoons.

1 teaspoon of the invention of oral liposomal glutathione reduced contains approximately 440 mg GSH.

A preferred mode sets a suggested dose based on body weight. Recommended amounts are for use in the treatment of influenza symptoms. For best results it is suggested that the invention be used at the early onset of flu symptoms of as a preventative after exposure the flu.

Gently stir liposomal glutathione into the liquid of your choice.

HIV and/or TB

Dose of liposomal reduced glutathione for individuals with HIV and/or TB is a range of ¼ teaspoon (containing 100 mg reduced glutathione) for every 30 pounds up to 100 pounds of weight. The dose over 100 pounds is a range of 1.0 teaspoon to 3.0 teaspoons twice a day. The preferred dose is 1.5 teaspoons twice a day orally in combination with the HIV drugs and/or the anti-tuberculosis drugs.

Heading

DETERMINE INDIVIDUAL DOSE BY BODY WEIGHT: For children

Under 30 lbs: ¼ teaspoon=100 mg GSH

30-60 lbs: ½ teaspoon=210 mg GSH

60-90 lbs: ¾ teaspoon=316 mg GSH

90-120 lbs: 1 teaspoon=422 mg GSH

120-150 lbs: 1½ teaspoon=630 mg GSH

Over 150 lbs: 1½ teaspoons=630 mg GSH

### Dosing Schedule for the Treatment of Acute and Chronic Symptoms of HHV-6 Virus Such as Chronic Fatigue

As stated, the initial dose should be according to body weight. For adults the dose is 1 and ½ teaspoon initially and repeat every 1 to 2 hours over 24 hour period. The amount and frequency of doses may be decreased as the individual begins to improve. The period of treatment will continue until severe symptoms are resolved. For chronic infections as seen with chronic fatigue syndrome, the present invention is continued at the level of 1 and ½ teaspoons twice a day until symptoms have abated. Ingestion of the liposomal preparation of reduced glutathione can result in a rapid reduction in viral symptoms as related in the examples cited. The mechanism may be related to one or more of the methods described. The rapid addition of reduced glutathione to the system by the invention has a number of avenues to facilitate restoration of normal general cell and immune cell function that results in the reduction of symptoms related to HHV-6 and virus infection in general.

### Dosing Schedule for the Treatment of Acute Symptoms of HIV+ Virus and Tuberculosis or Tuberculosis Alone or HHV-6 Virus or Other Virus Such as Encephalitis

As stated, the initial dose should be according to body weight. For adults the dose is 1 and ½ teaspoon initially and repeat every 1 to 2 hours over 24 hour period. The amount and frequency of doses may be decreased as the individual begins to improve. The period of treatment will continue until severe symptoms are resolved.

For chronic infections as seen with chronic fatigue syndrome, the present invention is continued at the level of 1 and ½ teaspoons twice a day until symptoms have abated.

Ingestion of the liposomal preparation of reduced glutathione can result in a rapid reduction in viral symptoms as related in the examples cited. The mechanism may be related to one or more of the methods described. The rapid addition of reduced glutathione to the system by the invention has a number of avenues to facilitate restoration of normal general cell and immune cell function that results in the reduction of symptoms related to HHV-6 and virus infection in general.

### Example 3

If the individual is not able to ingest oral medication the therapy is started with the intravenous infusion of glutathione in the following manner.

The solution used for intravenous administration is prepared with glutathione concentrations of 200 mg per cc. The material is stored in vials of 10 cc for a total of 2000 mg per vial. The infusion may consist of 600 mg to 2000 mg given by rapid push infusion through an intravenous line. The infusion may be repeated on an hourly or as needed basis lessen the flu symptoms.

Providing the intravenous glutathione in a concentration that provides physiologic osmolarity is important. Osmolarity is a measure of the osmotic pressure exerted by a solution across a perfect semi-permeable membrane. For instance, two identical solutions would have an osmolarity of zero. A solution that has twice as many particles on one side of a semi-permeable membrane as the other would have a higher osmolarity. The exact osmolarity of each solution would depend on the number of molecules or dissolved particles in the solution. In the body, we are looking at differences in the hundreds of milliosmoles, that is one-thousandth the concentration difference. Osmolarity is dependent on the number of particles in solution, but independent of the nature of the particles. The following table provides concentrations of glutathione in sterile water to create normal or hypertonic osmolarity. The average osmolarity of human serum is 290 mOsm. Solutions in the range of 240 to 340 mOsm are considered isotonic or roughly equivalent to the osmolarity of blood. Solutions that are hypotonic relative to cells have fewer dissolved solids or solutes than the interior of surrounding cells and results in fluid being pulled into cells. Thus, hypotonic fluids cause cells to swell and are considered dangerous to cells. Strategies for formulating concentrations of the fluids for intravenous infusion that create isotonic or hypertonic solutions are more desirable than using hypotonic solutions.

TABLE 1

| | Volume | milliOsmoles/ | Total |
|---|---|---|---|
| | in ml | ml | Milliosmoles |
| RLG 200 mg/ml | 8.00 | 1.89 | 15.12 |
| Sterile water | 12.00 | 0.00 | 0.00 |
| Total volume | 20.00 | | 15.12 |

Osmolarity: 856
RLG = Reduced L-Glutathione
For Glutathione 2000 mg

The infusion is continued at the rate of 2000 mg given over a period of 4 hours and repeated as needed on a continuous basis until the acute phase of the illness has resolved.
After the individual is able to resume oral ingestion of medications the oral liposomal encapsulation of reduced glutathione form of the invention is initiated at a rate of 400 mg., or one teaspoon every 2 hours.
Lower doses may be utilized over succeeding days until using the 1 and ½ teaspoon twice a day rate used for the long term therapy of non acute neurologic disease such as peripheral neuropathy described in the case example 2.

### Example 4

### Liposomal Glutathione Drink or Spray 2500 mg Per Ounce

| | % w/w |
|---|---|
| Deionized Water | 71.9 |
| Glycerin | 15.00 |
| Polysorbate-20 | 2.50 |
| Lecithin | 1.50 |
| Citrus Seed Extract | 0.50 |
| Potassium Sorbate | 0.10 |
| Glutathione | 8.50 |
| (reduced) | |

Components lecithin, ethyl alcohol, cholesterol and glycerin were commingled in a large volume flask and set aside for compounding (Alternatively, in all of the embodiments where the glutathione (reduced) percentage is 8.5, the glutathione (reduced percentage) can be lowered to 8.25 with 0.25% tocopherol acetate added). In that instance the table is (Example 5):

### Liposomal Glutathione Drink or Spray 2500 mg Per Ounce

| | % w/w |
|---|---|
| Deionized Water | 71.9 |
| Glycerin | 15.00 |
| Polysorbate-20 | 2.50 |
| Lecithin | 1.50 |
| Citrus Seed Extract | 0.50 |
| Potassium Sorbate | 0.10 |
| Glutathione | 8.25 |
| (reduced) | |

and optional 0.25% alpha-tocopherol.

### For Ancillary

In a separate beaker, water, hydroxy citric acid, glycerin, polysorbate 20, glutathione were mixed and heated to 50 degrees C.
The water mixture was added to the lipid mixture while vigorously mixing with a high speed, high shear homogenizing mixer at 750-1500 rpm for 30 minutes.
The homogenizer was stopped and the solution was placed on a magnetic plate, covered with parafilm and mixed with a magnetic stir bar until cooled to room temperature. Citrus seed extract were added and the solution was placed in appropriate dispenser for ingestion as a liquid or spray dispenser.
Analysis of the preparation under an optical light microscope with polarized light at 400× magnification confirmed presence of both multilamellar lipid vesicles (MLV) and unilamellar lipid vesicles.
The preferred embodiment includes the variations of the amount of glutathione to create less concentrated amounts of glutathione. The methods of manufacture described in Keller et al, U.S. Pat. No. 5,891,465 are incorporated into this description.
A variation of the preferred embodiment of the invention is the addition of EDTA (ethylene diamine tetraacetic acid) 100 mg per ounce to be encapsulated in the liposome along with the glutathione.

### Example 6

### Liposomal Glutathione Drink or Spray 2500 mg Per Ounce or Form Suitable for Encapsulation or Gel

| | % w/w |
|---|---|
| Deionized Water | 74.4 |
| Glycerin | 15.00 |
| Lecithin | 1.50 |
| Citrus Seed Extract | 0.50 |
| Potassium Sorbate | 0.10 |
| (optional spoilage retardant) | |
| Glutathione | 8.5 |
| (reduced) | |

### Example 7

| | % w/w |
|---|---|
| Deionized Water | 74.4 |
| Glycerin | 15.00 |
| Lecithin | 1.50 |
| Citrus Seed Extract | 0.50 |
| Potassium Sorbate | 0.10 |
| (optional spoilage retardant) | |
| Glutathione | 8.25 |
| (reduced) | |

A lipid mixture having components lecithin, ethyl alcohol and glycerin were commingled in a large volume flask and set aside for compounding.
In a separate beaker, a water mixture having water, glycerin, glutathione were mixed and heated to 50.degree. C.
The water mixture was added to the lipid mixture while vigorously mixing with a high speed, high shear homogenizing mixer at 750-1500 rpm for 30 minutes.
The homogenizer was stopped and the solution was placed on a magnetic stifling plate, covered with parafilm and mixed with a magnetic stir bar until cooled to room temperature. Normally, citrus seed extract would be added. Normally, a spoilage retardant such as potassium sorbate or BHT would be added. The solution would be placed in appropriate dispenser for ingestion as a liquid or administration as a spray.
However, in all examples given, and as shown in the table below, the formulation of liposomal glutathione can be formulated without the addition of potassium sorbate or citrus seed, or equivalent taste and/or preservative can be added so long as they do not impair the efficacy of the composition.
Analysis of the preparation under an optical light microscope with polarized light at 400× magnification confirmed presence of both multilamellar lipid vesicles (MLV) and unilamellar lipid vesicles.
The preferred embodiment includes the variations of the amount of glutathione to create less concentrated amounts of glutathione. The methods of manufacture described in Keller et al U.S. Pat. No. 5,891,465 are incorporated into this description.

### Example 8

### Liposomal Glutathione Drink or Spray 1000 mg Per Ounce

### With EDTA 1000 mg Per Ounce

| | % w/w |
|---|---|
| Deionized Water | 73.55 |
| Glycerin | 15.00 |
| Polysorbate-20 | 2.50 |
| Lecithin | 1.50 |
| Citrus Seed Extract | 0.50 |
| Tocopherol Acetate | 0.25 |
| Potassium Sorbate | 0.10 |
| Glutathione (reduced) | 3.30 |
| EDTA | 3.30 |

Embodiment two of the invention includes the incorporation of the fluid liposome (such as that prepared in Example 1A) into a gelatin based capsule to improve the stability, provide a convenient dosage form, and assist in sustained release characteristics of the liposome. The present embodiment relates to the use of glutathione in the reduced state encapsulated into liposomes or formulated as a preliposome formulation and then put into a capsule. The capsule can be a soft gel capsule capable of tolerating a certain amount of water, a two-piece capsule capable of tolerating a certain amount of water or a two-piece capsule where the liposomes are preformed then dehydrated.
The liposome-capsule unit containing biologically encapsulated material can be taken in addition to orally, used for topical unit-of-use application, or other routes of application such as intra-occular, intranasal, rectal, or vaginal.
The composition of examples 1 and 2 may be utilized in the encapsulated embodiment of this invention.
Gelatin capsules have a lower tolerance to water on their interior and exterior. The usual water tolerance for a soft gel capsule is 10% on the interior. The concentration of water in a liposome formulation can range from 60-90% water. An essential component of the present invention is the formulation of a liposome with a relatively small amount of water, in the range of 5-10%. By making the liposome in a low aqueous system, the liposome is able to encapsulate the biologically active material and the exposure of water to the inside lining of the capsule is limited. The concentration of water should not exceed that of the tolerance of the capsule for which it is intended. The preferred capsule for this invention is one that can tolerate water in the 15-20% range.
The method described by Keller et al, U.S. Pat. No. 6,726,924 are incorporated in this description.
Components are commingled and liposomes are made using the injection method (Lasic, D., Liposomes, Elsevier, 88-90, 1993). When liposome mixture cooled down 0.7 ml was drawn into a 1 ml insulin syringe and injected into the open-end of a soft gelatin capsule then sealed with tweezers. The resulting capsule contains 10 mg CoQ10. Filling of gel caps on a large scale is best with the rotary die method or others such as the Norton capsule machine.

### Example 9

### Glutathione LipoCap Formulation

| Ingredient | Concentration (%) |
|---|---|
| Sorbitan Oleate | 2.0 |
| Glutathione | 89.8 |
| Purified Water | 4.0 |
| Potassium Sorbate | 0.2 |
| Polysorbate 20 | 2.0 |
| Phospholipon 90 (DPPC) | 2.0 |

Components are commingled and liposomes are made using the injection method (Lasic, D., Liposomes, Elsevier, 88-90, 1993). When liposome mixture cooled down 0.7 ml was drawn into a 1 ml insulin syringe and injected into the open-end of a soft gelatin capsule then sealed with tweezers. The resulting one gram capsule contains 898 IU of Vitamin E. Large scale manufacturing methods for filling gel caps, such as the rotary die process, are the preferred method for commercial applications.

Embodiment number three of the present invention includes the creation of liposome suspension using a self-forming, thermodynamically stable liposomes formed upon the adding of a diacylglycerol-PEG lipid to an aqueous solution when the lipid has appropriate packing parameters and the adding occurs above the melting temperature of the lipid. The method described by Keller et al, U.S. Pat. No. 6,610,322 is incorporated into this description.
Most, if not all, known liposome suspensions are not thermodynamically stable. Instead, the liposomes in known suspensions are kinetically trapped into higher energy states by the energy used in their formation. Energy may be provided as heat, sonication, extrusion, or homogenization. Since every high-energy state tries to lower its free energy, known liposome formulations experience problems with aggregation, fusion, sedimentation and leakage of liposome associated material. A thermodynamically stable liposome formulation which could avoid some of these problems is therefore desirable.
The present embodiment prefers liposome suspensions which are thermodynamically stable at the temperature of formation. The formulation of such suspensions is achieved by employing a composition of lipids having several fundamental properties. First, the lipid composition must have packing parameters which allow the formation of liposomes. Second, as part of the head group, the lipid should include polyethyleneglycol (PEG) or any polymer of similar properties which sterically stabilizes the liposomes in suspension. Third, the lipid must have a melting temperature which allows it to be in liquid form when mixed with an aqueous solution.
By employing lipid compositions having the desired fundamental properties, little or no energy need be added when mixing the lipid and an aqueous solution to form liposomes. When mixed with water, the lipid molecules disperse and self assemble as the system settles into its natural low free energy state. Depending on the lipids used, the lowest free energy state may include small unilamellar vesicle (SUV) liposomes, multilamellar vesicle (MLV) liposomes, or a combination of SUVs and MLVs. In one aspect, the invention includes a method of preparing liposomes. The method comprises providing an aqueous solution; providing a lipid solution, where the solution has a packing parameter measurement of Pₐ (Pₐ references the surface packing parameter) between about 0.84 and 0.88, a Pᵥ (Pᵥ references the volume packing parameter) between about 0.88 and 0.93, (See, D. D. Lasic, Liposomes, From Physics to Applications, Elsevier, p. 51 1993), and where at least one lipid in the solution includes a polyethyleneglycol (PEG) chain; and combining the lipid solution and the aqueous solution. The PEG chain preferably has a molecular weight between about 300 Daltons and 5000 Daltons. Kinetic energy, such as shaking or vortexing, may be provided to the lipid solution and the aqueous solution. The lipid solution may comprise a single lipid. The lipid may comprise dioleolylglycerol-PEG-12, either alone or as one of the lipids in a mixture. The method may further comprise providing an active compound, in this case glutathione (reduced); and combining the active compound with the lipid solution and the aqueous solution.

### Treatment of HIV+ and Latent or Acute Tuberculosis:

Initially, for patient treatment, reference the recommended or suggested doses on the Drug Package insert for anti-retroviral treatment and administer that amount. For persons sensitive to the ARV drugs, due to the synergies between liposomal glutathione and ARV drugs, on a physician's recommendation, it may be possible to maintain the suggested liposomal glutathione doses above, but reduce the dose of the ARV drug to 70% of the recommended dose, 80% of the recommended dose, or 90% of the recommended dose. This reduced dosage would avoid some side effects resulting from many of the ARV drugs, including dyslipidemia.
Simultaneously, 3.3%, 4%, 5%, 6%, 7%, 7.5%, 8%, 8.5% or 9% lipoceutical glutathione may be used with anti-tuberculosis drugs. Initially, reference the recommended or suggested doses on the Drug Package insert for anti-tuberculosis treatment. For persons sensitive to the anti-tuberculosis drugs, due to the synergies between liposomal glutathione and anti-tuberculosis drugs, on a physician's recommendation, it may be possible to maintain the suggested liposomal glutathione doses above, but reduce the dose of the anti-tuberculosis drug to 70% of the recommended dose, 80% of the recommended dose, or 90% of the recommended dose.

With respect to combinations and methods with ARV drugs, the liposomal glutathione should preferably be at 8.0, 8.25, 8.5 or 9% w/w to maintain the synergy between the pharmaceutical compositions.

### Liposomal Glutathione in the Management of the Symptoms of Acute Viral Illness

### Case 1.

Chris T is a 37 year old man who presents with fatigue, weakness, diaphoresis, pallor and a sense of exhaustion. The symptoms had been present and progressing over a 14 day period of time, following an episode described as a “bad flu”. At the time of evaluation at 10 AM he was considering returning to bed as even light lifting tasks and standing as part of his sales job was exhausting.
600 mg of oral liposomal glutathione was administered and the individual observed. He noted that approximately 45 minutes after ingesting the invention his symptoms began to lessen. His color returned to normal, the diaphoresis ceased and he felt a significant return of energy and strength. The improvement lasted almost an hour when his symptoms began to return.
Chris T. repeated the 600 mg dose and 20 to 30 minutes later again felt resolution of his symptoms. He repeated this schedule every 1 to 2 hours through the day. By 8 PM he had ingested 1 and ½ ounces (approximately 3750 mg) of the invention and his symptoms had resolved completely. Using the invention through the day, he was able to complete his sales job, which on that day included standing all day, some light lifting of his product and interacting with customers continually through the day. The next morning in this example 2, Chris T., reported that his flu symptoms had abated.

### Liposomal Glutathione in the Management of Peripheral Neuropathy

### Case 2.

l. M. is a 79 year old woman with a history peripheral neuropathy affecting her legs that has been present for 10 years. The patient's neuropathy has prevented her from standing on hard surfaces due to the pain that activity induced. She used a wheelchair for shopping and was not able to stand on the hard ceramic tiles of her kitchen. L. M. initiated use of the invention in the form of oral liposomal glutathione at the rate of 1 and ½ teaspoons per day. She was using no other medications. After use of the invention for 8 weeks she began to notice a decrease in the pain. At 10 weeks she reported that she could again stand on her kitchen floor for the two hours that it required to cook a dinner.

### Liposomal Glutathione Antiviral Effect on HHV-6 Infected Cell Culture

HHV-6A is a cell associated virus; cell free virus is often not very infectious. Therefore, an assay was used that combined HHV-6 infected cells with uninfected cells. A study by a laboratory independent of the inventor commissioned by a group, the HHV-6 Foundation, which is also independent from the inventor were run. To various cultures of this mixture of cells, the submitted drugs, at various concentrations were added. The positive control was the cell combination with no drug and the negative control was uninfected cells only. After the assay was allowed to run for 7 days, a fluorometric cytoproliferation assay was run and all assay conditions were calculated as a percentage of the negative control. If a drug assay was at least 90% of the negative control, it was scored as being effective against HHV-6. A parallel cytotoxicity assay was run without infected cells to test whether the drugs are cytotoxic to the HSB 2 cells used in this experiment.
HHV-6A GS. Human herpesvirus 6A, strain GS is adapted for growth in tissue culture. HHV-6A is the strain most commonly reactivated in AIDS patients and in patients with multiple sclerosis. HSB-2, a human T-lymphoblastoid suspension cell line, was derived from the peripheral blood buffy coat of a patient with acute lymphoblastic leukemia and propagated as tumors in newborn Syrian hamsters.

### Controls:

Positive control—Cultures of infected and uninfected cells at a ratio of one infected cell for every four uninfected cells, no drugs or experimental reagents.
Negative control—cultures of uninfected cells only.
Cytotoxicity controls—drugs run at same concentrations as for the antiviral assay, but with uninfected cells only.
Drug comparison control: One plate was run with Foscarnet, Ganciclovir, and Cidofovir. In addition a Foscarnet comparison was run on each test drug plate.

Assay Parameters:

200 μL cultures, plated with 5×103 uninfected cells per culture plus 1.25×103 infected cells, if infected cells are present in the culture.
Four replicates were run for each Foscarnet, Ganciclovir and Cidofovir concentration on the comparison control plates (antiviral and cytotoxicity). In addition on each antiviral experimental drug plate there were duplicate wells of each Foscarnet concentration.
10 replicates were run at each concentration of each experimental drug in the antiviral assay and for the cytotoxicity controls 4 replicates were run for the experimental drugs.
Cells were allowed to grow for seven days at which time the experiment was terminated and the cytoproliferation assay was run.

### Cytotoxicity/Cytoproliferation Assay:

Fluorescent dye, 20 μL added to each culture.
Incubation for six hours at 37° C.
Read on a fluorometric reader at excitation of 530 nm, emission of 580 nm, and a gain of 35.

### Calculations:

Fluorometric readings for replicate cultures are averaged.
The average of the negative control is set at 100%, and the average of the other assay conditions are represented as a percentage of the negative control.

### Evaluation and Reporting of Results:

### Validity

This study is considered valid when the positive control shows evidence of viral infection (cytopathological effect) and is 65% or less of the negative control. The negative control should appear as a healthy growing culture by microscopic inspection.

### Report of Results

The final report contains the fluorometric readings for each culture, the average of replicate cultures, and each assay condition is presented as a percentage of the negative control. These data are presented in tabular form in an appendix.

A discussion of the data is presented.
Criteria for cytotoxicity: If the average of the cultures with drug but without virus is 85% of the negative control that concentration of drug is judged as not being cytotoxic. If the percentage is between 75% and 85% of the negative control is said to have slight cytotoxicity. Any value below 75% is scored as cytotoxic.
Criteria for antiviral efficacy: If the average growth for infected cultures at a specific drug concentration is over 90% of the negative control, the drug is scored as effective against HHV-6A. If the average is between 90% and 10% above the average for the positive control the drug at that concentration is scored as partially effective against the virus. If it is 5%-10% above the positive control it is scored as slightly effective. Scores within 5% of the positive control are judged as ineffective against the virus. Scores below 5% of the positive control are judged as being due to the cytotoxicity of the drug.
Study 1: In an initial study several drugs known to be effective against HHV-6 were evaluated for their efficacy. The material used included Foscarnet (phosphonoformic acid) Sigma P6801, Ganciclovir, Sigma G2536, Cidofovir (Vistide injection 75 mg/mL) Gilead Sci, Amantadine, Sigma A1260, Ribavirin, Sigma R9644, Doxycyline Hyclate, Sigma D9891, PBS 119, (Combination of chloroquine, verapamil, Dilantin and quercetin), Chloroquine diphosphate, Sigma C6628, Neem elixir, Glycyrrhizic acid, Sigma G2135, and Lithium carbonate, Sigma L4283.
Study 1 summary: Various drugs were tested in vitro to see if they suppress the propagation of HHV-6A GS into uninfected HSB-2 cells. Cultures with infected and uninfected cells were given various dosages of the drugs being tested and allowed to grow for 7 days. At the end of seven day a fluorometric cytoproliferation assay was preformed and the growth of uninfected cells (negative control) was compared to the growth of infected cells without drug (positive control) and the growth of the cells with the various drugs. Cytotoxicity controls were run with only uninfected cells and the drugs. No drug tested was able to suppress HHV-6 completely or better able to suppress viral propagation than Foscarnet.

### Study 2

Comparison control drug: Foscarnet (phosphonoformic acid) Sigma P6801
Experimental drugs: Nexavir (Kutapressin), L-Lysine, Sigma L9037, Gabapentin (Neurotonin), Sigma G154, Lovenex (Heparin, Enoxaparin Sodium) Compound X from Company X, Oleuropein (Olive Leaf Extract), ImmunoPro, (non-denatured whey protein), Lactoferrin from bovine milk, Sigma L9507, COMPOUND X (Company X substance A000556500), Lipoceutical Glutathione™ (Readisorb Products, Your Energy Systems, Inc., 555 Bryant St., #305, Palo Alto, Calif. 94301), Resveratrol, Sigma R5010, FW 228.2.
Percentage increase over positive (infected) control at optimal dosage: Foscarnet 20%. Lipoceutical Glutathione™ 30%.
Additional testing is being performed to determine optimal effective range and no cytotoxicity was found for Lipoceutical Glutathione™.
Conclusion: Of the twenty compounds tested, Lipoceutical Glutathione™, the trade name of the present liposomal glutathione invention, showed efficacy against HHV-6 virus. The study also demonstrated that there was no cytotoxicity from liposomal formulated reduced glutathione.

Heading

### Case Examples and Dosing

E. W. a 48 year old woman who has experienced severe fatigue symptoms for over 20 years. She relates that while her symptoms developed after exposure to paints and solvent exposure, there was no clear toxin identified and a chronic viral component has been suspected.
Recently, the symptom complex had expanded to include loose stools that had been present for one month. E. W. started liposomal glutathione at 1 and ½ teaspoons once a day in 3 divided doses. After a week of use she noted that her stools had become more firm. After a month of use, her stools became normal.
At the time of this report, the individual had been using the liposomal glutathione for 3 months. She noted that she had more stamina, although she was not yet able to return to work. At the same time she was able to tolerate emotional stresses that normally would have caused a significant setback and prolonged exhaustion.
E. W. reports that since using the liposomal glutathione she is functioning significantly better than she has since developing the chronic fatigue symptoms.

Heading

### REFERENCES

[Para 1] Ablashi D V, Bernbaum J, DiPaolo J A. Human herpesvirus 6 as a potential copathogen. Trends in microbiology. 1995 August; 3(8):324-7. Review. PMID: 8528618
[Para 2] Akira S. The role of IL-18 in innate immunity. Current Opinion Immunology. 2000 February; 12(1):59-63. PMID: 10679398.
[Para 3] Awasthi V D, Goins B, Klipper R, Phillips W T. Dual radiolabeled liposomes: biodistribution studies and localization of focal sites of infection in rats. Nuclear Medicine and Biology. 1998 February; 25(2):155-60. PMID: 9468030.
[Para 4] Awasthi V D, Goins B, Klipper R, Phillips W T. Accumulation of PEG-liposomes in the inflamed colon of rats: potential for therapeutic and diagnostic targeting of inflammatory bowel diseases. Journal Drug Targets. 2002 August; 10(5):419-27. PMID: 12442813 Bauer G. Reactive oxygen and nitrogen species: efficient, selective, and interactive signals during intercellular induction of apoptosis. Anticancer Res. 2000 November-December; 20(6B):4115-39. Review. PMID: 11205238
[Para 5] Bender A S, Reichelt W, Norenberg M D. Characterization of cystine uptake in cultured astrocytes. Neurochemistry International 2000; 37(2-3):269-76. PMID: 10812212
[Para 6] Boya P, de la Pena A, Beloqui O, Larrea E, Conchillo M, Castelruiz Y, Civeira M P, Prieto J. Antioxidant status and glutathione metabolism in peripheral blood mononuclear cells from patients with chronic hepatitis C. Journal of Hepatology. 1999 November; 31(5):808-14. PMID: 10580577
[Para 7] Braun D K, Dominguez G, Pellett P E. Human herpesvirus 6. Clinical microbiology reviews. 1997 July; 10(3):521-67. Review. PMID: 9227865
[Para 8] Buhl R, Jaffe H A, Holroyd K J, Wells F B, Mastrangeli A, Saltini C, Cantin A M, Crystal R G. Systemic glutathione deficiency in symptom-free HIV-seropositive individuals. Lancet. 1989 Dec. 2; 2(8675):1294-8. PMID: 2574255
[Para 9] Cai J, Chen Y, Seth S, Furukawa S, Compans R W, Jones D P. Inhibition of influenza infection by glutathione. Free radical biology & medicine. 2003 Apr. 1; 34(7):928-36. PMID: 12654482
[Para 1 0] Cattaneo R. Four viruses, two bacteria, and one receptor: membrane cofactor protein (CD46) as pathogens' magnet. Journal of Virology. 2004 May; 78(9):4385-8. PMID: 15078919
[Para 11] Constantinescu I, Liposomes and blood cells: a flow cytometric study. Artificial cells, blood substitutes, and immobilization biotechnology, 2003 November; 31(4):395-424. PMID: 14672416
[Para 12] Cruz-Aguado R, Francis-Turner L, Diaz C M, Antunez I. Quinolinic acid lesion induces changes in rat striatal glutathione metabolism. Neurochemistry international. 2000 July; 37(1):53-60. PMID: 10781845
[Para 13] Cunningham A L, Naif H, Saksena N, Lynch G, Chang J, Li S, Jozwiak R, Alali M, Wang B, Fear W, Sloane A, Pemberton L, Brew B. HIV infection of macrophages and pathogenesis of AIDS dementia complex: interaction of the host cell and viral genotype. Journal Leukocyte Biology 1997 July; 62(1):117-25. PMID: 9226002
[Para 14] Dattwyler R J, Thomas J A, Benach J L, Golightly M G. Cellular immune response in Lyme disease: the response to mitogens, live Borrelia burgdorferi, NK cell function and lymphocyte subsets. Zentralblatt fur Bakteriologie, Mikrobiologie und Hygiene. l. Abt. Originale A, Medizinische Mikrobiologie, Infektionskrankheiten und Parasitologie=International journal of microbiology and hygiene. A, Medical microbiology, infectious. 1986 December; 263(1-2):151-9. PMID: 2953150
[Para 15] De Bolle L, Naesens L, De Clercq E. Update on human herpesvirus 6 biology, clinical features, and therapy. Clinical microbiology reviews. 2005 January; 18(1):217-45. PMID: 15653828
[Para 16] Dockrell D. Human herpesvirus 6: molecular biology and clinical features. Journal Medical Microbiology 2003 52:5-18 PMID: 12488560
[Para 17] Droge, W., Eck, H. P., Gmunder, H. and Mihm, S. (1991) Requirement for prooxidant and antioxidant states in T cell mediated immune responses.—Relevance for the pathogenetic mechanisms of AIDS? Klin. Wochenschr. 69, 1118-1122 PMID: 1798289
[Para 18] Dringen R, Gutterer J M, Hirrlinger J. Glutathione metabolism in brain metabolic interaction between astrocytes and neurons in the defense against reactive oxygen species. European journal of biochemistry/FEBS. 2000 August; 267(16):4912-6. Review. PMID: 10931173
[Para 19] Flamand L, Gosselin J, D'Addario M, Hsicott J, Ablashi D V, Gallo R C, Menezes J. Human herpesvirus 6 induces interleukin-1 beta and tumor necrosis factor alpha, but not interleukin-6, in peripheral blood mononuclear cell cultures. Journal Virology. 1991 September; 65(9):5105-10 PMID: 1651426
[Para 20] Folks T M, Clouse K A, Justement J, Rabson A, Duh E, Kehrl J H, Fauci S A. Tumor necrosis factor α induces expression of human immunodeficiency virus in a chronically infected T-cell clone. Proceedings National Academy of Sciences. USA 1989; 86:2365=2368. PMID: 2784570
[Para 21] Garaci E, Palamara A T, Di Francesco P, Favalli C, Ciriolo M R, Rotilio G. Glutathione inhibits replication and expression of viral proteins in cultured cells infected with Sendai virus. Biochemical and biophysical research communications. 1992 Nov. 16; 188(3):1090-6. PMID: 1332709
[Para 22] Garaci E, Palamara A T, Ciriolo M R, D'Agostini C, Abdel-Latif M S, Aquaro S, Lafavia E, Rotilio G. Intracellular GSH content and HIV replication in human macrophages. Journal Leukocyte Biology 1997 July; 62(1):54-9. PMID: 9225993
[Para 23] Garcia-Monco J C, Benach J L. Mechanisms of injury in Lyme neuroborreliosis.
Semin Neurol. 1997 March; 17(1):57-62. Review. PMID: 9166961
[Para 24] Gomez M I, Sokol S H, Muir A B, Soong G, Bastien J, Prince A S. Bacterial Induction of TNF-{alpha} Converting Enzyme Expression and IL-6 Receptor {alpha} Shedding Regulates Airway Inflammatory Signaling. Journal Immunology. 2005 Aug. 1; 175(3):1930-6. PMID: 16034137
[Para 25] Hothersall J S, Cunha F Q, Neild G H, Norohna-Dutra A A. Induction of nitric oxide synthesis in J774 cells lowers intracellular glutathione: effect of modulated glutathione redox status on nitric oxide synthase induction. Biochem J. 1997 Mar. 1; 322 (Pt 2):477-81. PMID: 9065766
[Para 26] Holmes G P, Kaplan J E, Gantz N M, Komaroff A L, Schonberger L B, Straus S E, Jones J F, Dubois R E, Cunningham-Rundles C, Pahwa S, Chronic fatigue syndrome: A working case definition. Annals of Internal Medicine. 1988 March; 108(3):387-9. Division of Viral Diseases, Centers for Disease Control, Atlanta, Ga. PMID: 2829679 Inoue Y, Yasukawa M, Fujita S. Induction of T-cell apoptosis by human herpesvirus 6. Journal of Virology. 1997 May; 71(5):3751-9. PMID: 9094650
[Para 27] Herzenberg, L. A., S. C. De Rosa, J. G. Dubs, M. Roederer, M. T. Anderson, S. W. Ela, S. C. Deresinski, and L. A. Herzenberg. 1997. Glutathione deficiency is associated with impaired survival in HIV disease. Proc. Natl. Acad. Sci. USA 94:1967-1972
[Para 28] Ishii Y, Partridge C A, Del Vecchio P J, Malik A B. Tumor necrosis factor-alpha-mediated decrease in glutathione increases the sensitivity of pulmonary vascular endothelial cells to H202. Journal Clinical Investigation 1992 March; 89(3):794-802. PMID: 1541673
[Para 29] James S J, Cutler P, Melnyk S, Jernigan S, Janak L, Gaylor D, and Neubrander J, Metabolic biomarkers of increased oxidative stress and impaired methylation capacity in children with autism, The American journal of clinical nutrition 2004; 80:1611-7; PMID: 15585776.
[Para 30] Johansen J S, Harris A K, Rychly D J, Ergul A. Oxidative stress and the use of antioxidants in diabetes: Linking basic science to clinical practice. Cardiovascular Diabetology 2005, 4:5 PMID: 15862133
[Para 31] Kalebic T, Kinter A, Poli G, Anderson M E, Meister A, Fauci A S. Suppression of human immunodeficiency virus expression in chronically infected monocytic cells by glutathione, glutathione ester, and N-acetylcysteine. Proceedings of the National Academy of Sciences of the United States of America. 1991 Feb. 1; 88(3):986-90. PMID: 1704137
[Para 32] Krueger G, Ablashi D. Human Herpesvirus-6: A short Review of its Biological Behavior. Intervirology. 2003; 46(5):257-69. PMID: 14555846
[Para 33] Kurita-Taniguchi M, Fukui A, Hazeki K, Hirano A, Tsuji S, Matsumoto M, Watanabe M, Ueda S, Seya T. Functional modulation of human macrophages through CD46 (measles virus receptor): production of IL-12 p40 and nitric oxide in association with recruitment of protein-tyrosine phosphatase SHP-1 to CD46. Journal of Immunology. 2000 Nov. 1; 165(9):5143-52. PMID: 11046046
[Para 34] Kyto V, Lapatto R, Lakkisto P, Saraste A, Voipio-Pulkki L M, Vuorinen T, Pulkki K. Glutathione depletion and cardiomyocyte apoptosis in viral myocarditis. European Journal of Clinical Investigation. 2004 March; 34(3):167-75. PMID: 15025674
[Para 35] Lusso, P., Markham, P. D., Tschachler, E., di Marzo Veronese, F., Salahuddin, S. Z., Ablashi, D. V., Pahwa, S., Krohn, K. & Gallo, R. C. In vitro cellular tropism of human B-lymphotropic virus (human herpesvirus-6). Journal of Experimental Medicine 167, 1659-1670. PMID: 3259254
[Para 36] Mayne M, Cheadle C, Soldan S S, Cermelli C, Yamano Y, Akhyani N, Nagel J E, Taub D D, Becker K G, Jacobson S. Gene Expression Profile of Herpesvirus-Infected T Cells Obtained Using Immunomicroarrays: Induction of Proinflammatory Mechanisms. Journal of Virology. 2001 December; 75(23): 11641-11650. PMID: 11689646
[Para 37] Mondal D, Pradhan L, Ali M, Agrawal K C. HAART drugs induce oxidative stress in human endothelial cells and increase endothelial recruitment of mononuclear cells: exacerbation by inflammatory cytokines and amelioration by antioxidants.
Cardiovasc Toxicol. 2004; 4(3):287-302. PMID: 15470276
[Para 38] Montessori V, Press N, Harris M, Akagi L, Montaner J S Adverse effects of antiretroviral therapy for HIV infection. Canadian Medical Association journal. 2004 Jan. 20; 170(2): 229-238. PMCID: 315530
[Para 39] Nicolson G L, Gan R, Haier J. Multiple co-infections (Mycoplasma, Chlamydia, human herpes virus-6) in blood of chronic fatigue syndrome patients:
association with signs and symptoms. Acta pathologica, microbiologica, et immunologica Scandinavica. 2003 May; 111(5):557-66. PMID: 12887507
[Para 40] Nucci C, Palamara A T, Ciriolo M R, Nencioni L, Savini P, D'Agostini C, Rotilio G, Cerulli L, Garaci E. Imbalance in corneal redox state during herpes simplex virus 1-induced keratitis in rabbits. Effectiveness of exogenous glutathione supply. Experimental Eye Research. 2000 February; 70(2):215-20. PMID: 106551470′Connor E, Devesa A, Garcia C, Puertes I R, Oellin A, Vina J R.
Biosynthesis and maintenance of GSH in primary astrocytes cultures: role of L-cystine 680(1-2):157-63. PMID: 7663972
[Para 41] Osterholm M T. Preparing for the Next Pandemic. New England Journal of Medicine 2005; 352(18):1839-42. PMID: 15872196
[Para 42] Palamara A T, Perno C F, Ciriolo M R, Dini L, Balestra E, D'Agostini C, Di Francesco P, Favalli C, Rotilio G, Garaci E. Evidence for antiviral activity of glutathione: in vitro inhibition of herpes simplex virus type 1 replication. Antiviral Research. 1995 June; 27(3):237-53. PMID: 8540746
[Para 43] Palamara A T, Garaci E, Rotilio G, Ciriolo M R, Casabianca A, Fraternale A, Rossi L, Schiavano G F, Chiarantini L, Magnani M. Inhibition of murine AIDS by reduced glutathione. AIDS research and human retroviruses. 1996 Sep. 20; 12(14):1373-81. PMID: 8891117
[Para 44] Pancewicz S A, Skrzydlewska E, Hermanowska-Szpakowicz T, Zajkowska J M, Kondrusik M. Role of reactive oxygen species (ROS) in patients with erythema migrans, an early manifestation of Lyme borreliosis. Medical science monitor: international medical journal of experimental and clinical research. 2001 November-December; 7(6):1230-5. PMID: 11687735
[Para 45] Pancewicz S A, Hermanowska-Szpakowicz T, Makarewicz-Plonska M, Witek A, Farbiszewski R, Zajkowska J, Michalska B. Decreased antioxidant-defence mechanisms in cerebrospinal fluid (CSF) in patients with tick-borne encephalitis (TBE). Neurol Neurochir Pol. 2002 July-August; 36(4):767-76. Polish. PMID: 12418140
[Para 46] Patnaik M, Komaroff A L, Conley E, Ojo-Amaize F A, Peter J B. Prevalence of IgM antibodies to human herpesvirus 6 early antigen (p41/38) in patients with chronic fatigue syndrome. The Journal of infectious diseases. 1995 November; 172(5): 1364-7. PMID: 7594679
[Para 47] Peterson J D, Herzenberg L A, Vasquez K, Waltenbaugh C. Glutathione levels in antigen-presenting cells modulate Th1 versus Th2 response patterns. Proc Natl Acad Sci USA. 1998 Mar. 17; 95(6):3071-6.
PMID: 9501217
[Para 48] Russler S K, Tapper M A, Knox K K, Liepins A, Carrigan D R. Pneumonitis associated with coinfection by human herpesvirus 6 and Legionella in an immunocompetent adult. The American journal of pathology. 1991 June; 138(6):1405-11. PMID: 1647138
[Para 49] Salahuddin S Z, Ablashi D V, Markham P D, Josephs S F, Sturzenegger S, Kaplan M, Halligan G, Biberfeld P, Wong-Staal F, Kramarsky B, et al. Isolation of a new virus, HBLV, in patients with lymphoproliferative disorders. Science. 1986 October d31; 234(4776):596-601. PMID: 2876520
[Para 50] Santoro F, Kennedy P E, Locatelli G, Malnati M S, Berger E A, Lusso P. CD46 is a cellular receptor for human herpesvirus 6. Cell. 1999 Dec. 23; 99(7):817-27. PMID: 10619434
[Para 51] Schmidt J, Metselaar J M, Wauben M H, Toyka K V, Storm G, Gold R. Drug targeting by long-circulating liposomal glucocorticosteroids increases therapeutic efficacy in a model of multiple sclerosis. Brain. 2003 August; 126(Pt 8):1895-904. Epub 2003 Jun. 4. PMID: 12805101
[Para 52] Shikuma C M, Valcour V G, Ratto-Kim S, Williams A E, Souza S, Gerschenson M, Day L, Kim J H, Shiramizu B. HIV-associated wasting in the era of highly active antiretroviral therapy: a syndrome of residual HIV infection in monocytes and macrophages? Clin Infect Dis. 2005 Jun. 15; 40(12):1846-8. Epub 2005 May 3. PMID: 15909275.
[Para 53] Soldan S S, Berti R, Salem N, Decchiero P, Flamand L, Calabresi P A, Brennan M B, Maloni H W, McFarland H F, Lin H C, Oatnaik M, Jacobson S. Association of human herpes virus 6 (HHV-6) with multiple sclerosis: increased IgM response to HHV-6 early antigen and detection of serum HHV-6 DNA. Nature Medicine. 1997 December; 3(12):1394-7. PMID: 9396611
[Para 54] St'astny F, Lisy V, Mares V, Lisa V, Balcar V J, Santamaria A. Quinolinic acid induces NMDA receptor-mediated lipid peroxidation in rat brain microvessels. Redox report: communications in free radical research. 2004; 9(4):229-33. PMID: 15479567
[Para 55] Sugaya N, Yoshikawa T, Miura M, Ishizuka T, Kawakami C, Asano Y. Influenza encephalopathy associated with infection with human herpesvirus 6 and/or human herpesvirus 7. Clinical infectious diseases: an official publication of the Infectious Diseases Society of America 2002 Feb. 15; 34(4):461-6. Epub 2002 Jan 4. PMID: 11797172
[Para 56] Uchida A. The cytolytic and regulatory role of natural killer cells in human neoplasia. Biochimica et biophysica acta. 1986 Dec. 17; 865(3):329-40. Review. No abstract available. PMID: 2947630.
[Para 57] Van Rooijen N, The liposome-mediated macrophage ‘suicide’ technique. Journal of Immunological Methods. 1989 Nov. 13; 124(1):1-6. PMID: 2530286
[Para 58] Venketaraman V, Dayaram Y K, Amin A G, Ngo R, Green R M, Talaue M T, Mann J, Connell N D. Role of glutathione in macrophage control of mycobacteria. Infection and Immunity. 2003 April; 71(4):1864-71. PMID: 12654802
[Para 59] Vogel J U, Cinatl J, Dauletbaev N, Buxbaum S, Treusch G, Cinatl J Jr, Gerein V, Doerr H W. Effects of S-acetylglutathione in cell and animal model of herpes simplex virus type 1 infection. Medical microbiology and immunology (Berl). 2005 January; 194(1-2):55-9. Epub 2003 Nov. 18. PMID: 14624358
[Para 60] Wallace H L 2nd, Natelson B, Gause W, Hay J. Human herpesviruses in chronic fatigue syndrome. Clinical and Diagnostic Laboratory Immunology, March 1999, p. 216-223, Vol. 6, No. 2 PMID: 10066657
[Para 61] Witschi A, Reddy S, Stofer B, Lauterburg B H The systemic availability of oral glutathione European journal of clinical pharmacology. 1992; 43(6):667-9. PMID: 1362956
[Para 62] Witschi A, Junker E, Schranz C, Speck R F, Lauterburg B H. Supplementation of N-acetylcysteine fails to increase glutathione in lymphocytes and plasma of patients with AIDS. AIDS research and human retroviruses. 1995 January; 11(1):141-3. PMID: 7734187
[Para 63] Yoshikawa T, Asano Y. Central nervous system complications in human herpesvirus-6 infection. Brain Development. 2000 August; 22(5):307-14. Review. PMID: 10891638

What is claimed is:

1. A pharmaceutical composition for treatment of tuberculosis, in a mammalian patient, comprising:
reduced glutathione in a liposomal formulation for oral administration having a concentration of said reduced glutathione in liposomes of said liposomal formulation of 8.25% w/w, and
at least one anti-tuberculosis pharmaceutical composition.

2. A pharmaceutical composition for treatment of tuberculosis, in a mammalian patient, comprising:
reduced glutathione in a liposomal formulation having a concentration of said reduced glutathione in liposomes of said liposomal formulation of 8.25% w/w;
at least one anti-tuberculosis pharmaceutical composition; and
a sterile diluent for intravenous administration of said liposomal formulation.

3. A pharmaceutical composition for treatment of an HIV+ mammalian patient having tuberculosis, comprising:
reduced glutathione in a liposomal formulation capable of oral administration;
at least one Anti-Retroviral Therapy having at least one pharmaceutical composition selected from the group of Nucleoside/tide Reverse Transcriptase Inhibitors (NRTIs), Protease Inhibitors (PIs), Non-nucleoside Reverse Transcriptase Inhibitors (NnRTIs); and
and at least one anti-tuberculosis pharmaceutical composition.

4. A pharmaceutical composition for treatment of an HIV+ mammalian patient having tuberculosis, comprising:
reduced glutathione in a liposomal formulation;
at least one Anti-Retroviral Therapy having at least one pharmaceutical composition selected from the group of Nucleoside/tide Reverse Transcriptase Inhibitors (NRTIs), Protease Inhibitors (PIs), Non-nucleoside Reverse Transcriptase Inhibitors (NnRTIs);
and at least one anti-tuberculosis pharmaceutical composition; and
a sterile diluent for intravenous administration of said liposomal formulation.

5. A pharmaceutical composition for treatment of an HIV+ mammalian patient having tuberculosis, comprising:
reduced glutathione in a liposomal formulation capable of oral administration;
at least one Anti-Retroviral Therapy having at least one pharmaceutical composition selected from the group of protease inhibitors (PI's), immune-base therapies, Pharmacokinetic Enhancers, Fusion Inhibitors, Entry Inhibitors, CCR5 co-receptor antagonists, HIV integrase strand transfer inhibitors, Integrase Inhibitors, or Maturation Inhibitors; and
and at least one anti-tuberculosis pharmaceutical composition.

6. A pharmaceutical composition for treatment of an HIV+ mammalian patient having tuberculosis, comprising:
reduced glutathione in a liposomal formulation;
at least one Anti-Retroviral Therapy having at least one pharmaceutical composition selected from the group of protease inhibitors (PI's), immune-base therapies, Pharmacokinetic Enhancers, Fusion Inhibitors, Entry Inhibitors, CCR5 co-receptor antagonists, HIV integrase strand transfer inhibitors, Integrase Inhibitors, or Maturation Inhibitors;
and at least one anti-tuberculosis pharmaceutical composition; and
a sterile diluent for intravenous administration of said liposomal formulation.

7. A method for enhancing immunological resistance to Mycobacterium Tuberculosis, in mammalian patients, comprising:
administering reduced glutathione orally in a liposomal formulation having a concentration of said reduced glutathione in liposomes of said liposomal formulation of 8.25% w/w to said patient displaying symptoms of tuberculosis.

8. A method for enhancing immunological resistance to Mycobacterium Tuberculosis, in mammalian patients, comprising:
administering reduced glutathione in a liposomal formulation having a concentration of said reduced glutathione in liposomes of said liposomal formulation 8.25% w/w; and
adding a sterile diluent for administration of said liposomal formulation to said patient.

9. A method for enhancing the efficacy of anti-viral treatment regimes in HIV+ patients having tuberculosis, comprising:
administering reduced glutathione orally in a liposomal formulation to said patient; and
administering at least one Anti-Retroviral Therapy having at least one pharmaceutical composition selected from the group of Nucleoside/tide Reverse Transcriptase Inhibitors (NRTIs), Protease Inhibitors (PIs), and Non-nucleoside Reverse Transcriptase Inhibitors (NnRTIs; and
administering at least one anti-tuberculosis drug.

10. A method for enhancing the efficacy of anti-viral treatment regimes in HIV+ patients having tuberculosis, comprising:
administering reduced glutathione in a liposomal formulation to said patient;
administering at least one Anti-Retroviral Therapy having at least one pharmaceutical composition selected from the group of Nucleoside/tide Reverse Transcriptase Inhibitors (NRTIs), Protease Inhibitors (PIs), and Non-nucleoside Reverse Transcriptase Inhibitors (NnRTIs;
administering at least one anti-tuberculosis drug; and
adding a sterile diluent for administration of said liposomal formulation to said patient.

11. A method for enhancing the efficacy of anti-viral treatment regimes in HIV+ patients, for mammalian patients, comprising:
administering reduced glutathione orally in a liposomal formulation to said patient;
administering at least one Anti-Retroviral Therapy having at least one pharmaceutical composition selected from the group of selected from the group of protease inhibitors (PI's), immune-base therapies, Pharmacokinetic Enhancers, Fusion Inhibitors, Entry Inhibitors, CCR5 co-receptor antagonists, HIV integrase strand transfer inhibitors, Integrase Inhibitors, or Maturation Inhibitors; and
administering at least one anti-tuberculosis drug.

12. A method for enhancing the efficacy of anti-viral treatment regimes in HIV+ patients, for mammalian patients, comprising:
administering reduced glutathione in a liposomal formulation to said patient;
administering at least one Anti-Retroviral Therapy having at least one pharmaceutical composition selected from the group of selected from the group of protease inhibitors (PI's), immune-base therapies, Pharmacokinetic Enhancers, Fusion Inhibitors, Entry Inhibitors, CCR5 co-receptor antagonists, HIV integrase strand transfer inhibitors, Integrase Inhibitors, or Maturation Inhibitors;
administering at least one anti-tuberculosis drug; and
adding a sterile diluent for administration of said liposomal formulation to said patient.
